(19) United States
(12) Patent Application Publication
(10) Pub. No.: US 2002/0010137 A1
(43) Pub. Date: Jan. 24, 2002

(54) Methods and compositions for inhibiting neoplastic cell growth

(75) Inventors: Avi Ashkenazi, San Mateo, CA (US); Audrey Goddard, San Francisco, CA (US); Austin L. Gurney, Belmont, CA (US); Robert D. Klein, Palo Alto, CA (US); Mary Napier, Hillsborough, CA (US); William I. Wood, Hillsborough, CA (US); Jean Yuan, San Mateo, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 09/828,366

(22) Filed: Apr. 5, 2001

Related U.S. Application Data
(63) Continuation of application No. 09/565,278, filed on Apr. 27, 2000.
(60) Provisional application No. 60/059,263, filed on Sep. 18, 1997.
(60) Provisional application No. 60/059,836, filed on Sep. 24, 1997.
(60) Provisional application No. 60/063,561, filed on Oct. 28, 1997.
(60) Provisional application No. 60/064,248, filed on Nov. 3, 1997.
(60) Provisional application No. 60/079,124, filed on Mar. 23, 1998.
(60) Provisional application No. 60/081,569, filed on Apr. 13, 1998.
(60) Provisional application No. 60/099,803, filed on Sep. 10, 1998.
(60) Provisional application No. 60/104,080, filed on Oct. 13, 1998.
(60) Provisional application No. 60/123,957, filed on Mar. 12, 1999.
(60) Provisional application No. 60/131,445, filed on Apr. 28, 1999.
(60) Provisional application No. 60/144,758, filed on Jul. 20, 1999.
(60) Provisional application No. 60/145,698, filed on Jul. 26, 1999.

(30) Foreign Application Priority Data
Aug. 28, 1998 (US) PCT/US98/17888
Sep. 10, 1998 (US) PCT/US98/18824
Sep. 16, 1998 (US) PCT/US98/19330
Mar. 19, 1999 (US) PCT/US99/06098
Sep. 8, 1999 (US) PCT/US99/20594
Sep. 15, 1999 (US) PCT/US99/21090
Oct. 5, 1999 (US) PCT/US99/23089
Nov. 30, 1999 (US) PCT/US99/28313
Dec. 2, 1999 (US) PCT/US99/28564
Dec. 20, 1999 (US) PCT/US99/30999
Jan. 5, 2000 (US) PCT/US00/00219
Jan. 6, 2000 (US) PCT/US00/00277
Feb. 22, 2000 (US) PCT/US00/04414
Mar. 2, 2000 (US) PCT/US00/05841
Mar. 10, 2000 (US) PCT/US00/06319
Mar. 15, 2000 (US) PCT/US00/06884
Dec. 1, 2000 (US) PCT/US00/32678

(51) Int. Cl. A61K 38/17; A61K 31/00

(52) U.S. Cl. 514/12; 514/1

(57) ABSTRACT

The present invention concerns methods and compositions for inhibiting neoplastic cell growth. In particular, the present invention concerns antitumor compositions and methods for the treatment of tumors. The invention further concerns screening methods for identifying growth inhibitory, e.g., antitumor compounds.

### FIELD OF THE INVENTION

The present invention concerns methods and compositions for inhibiting neoplastic cell growth. In particular, the present invention concerns antitumor compositions and methods for the treatment of tumors. The invention further concerns screening methods for identifying growth inhibitory, e.g., antitumor compounds.

### BACKGROUND OF THE INVENTION

Malignant tumors (cancers) are the second leading cause of death in the United States, after heart disease (Boring et al., CA Cancel J. Clin., 43:7 (1993)).

Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites (metastasis). In a cancerous state a cell proliferates under conditions in which normal cells would not grow. Cancer manifests itself in a wide variety of forms, characterized by different degrees of invasiveness and aggressiveness.

Despite recent advances in cancer therapy, there is a great need for new therapeutic agents capable of inhibiting neoplastic cell growth. Accordingly, it is the objective of the present invention to identify compounds capable of inhibiting the growth of neoplastic cells, such as cancer cells.

### SUMMARY OF THE INVENTION

The present invention relates to methods and compositions for inhibiting neoplastic cell growth. More particularly, the invention concerns methods and compositions for the treatment of tumors, including cancers, such as breast, prostate, colon, lung, ovarian, renal and CNS cancers, leukemia, melanoma, etc., in mammalian patients, preferably humans.

In one aspect, the present invention concerns compositions of matter useful for the inhibition of neoplastic cell growth comprising an effective amount of a PRO211, PRO228, PRO538, PRO172 or PRO182 polypeptide as herein defined, or an agonist thereof, in admixture with a pharmaceutically acceptable carrier. In a preferred embodiment, the composition of matter comprises a growth inhibitory amount of a PRO211, PRO228, PRO538, PRO172 or PRO182 polypeptide, or an agonist thereof. In another preferred embodiment, the composition comprises a cytotoxic amount of a PRO211, PRO228, PRO538, PRO172 or PRO182 polypeptide, or an agonist thereof. Optionally, the compositions of matter may contain one or more additional growth inhibitory and/or cytotoxic and/or other chemotherapeutic agents.

In a further aspect, the present invention concerns compositions of matter useful for the treatment of a tumor in a mammal comprising a therapeutically effective amount of a PRO211, PRO228, PRO538, PRO172 or PRO182 polypeptide as herein defined, or an agonist thereof. The tumor is preferably a cancer.

In another aspect, the invention concerns a method for inhibiting the growth of a tumor cell comprising exposing the cell to an effective amount of a PRO211, PRO228, PRO538, PRO172 or PRO182 polypeptide as herein defined, or an agonist thereof. In a particular embodiment, the agonist is an anti-PRO211, anti-PRO228, anti-PRO538, anti-PRO172 or anti-PRO182 agonist antibody. In another embodiment, the agonist is a small molecule that mimics the biological activity of a PRO211, PRO228, PRO538, PRO172 or PRO182 polypeptide. The method may be performed in vitro or in vivo.

In a still further embodiment, the invention concerns an article of manufacture comprising:

a container; and

a composition comprising an active agent contained within the container; wherein the composition is effective for inhibiting the neoplastic cell growth, e.g., growth of tumor cells, and the active agent in the composition is a PRO211, PRO228, PRO538, PRO172 or PRO182 polypeptide as herein defined, or an agonist thereof. In a particular embodiment, the agonist is an anti-PRO211, anti-PRO228, anti-PRO538, anti-PRO172 or anti-PRO182 agonist antibody. In another embodiment, the agonist is a small molecule that mimics the biological activity of a PRO211, PRO228, PRO538, PRO172 or PRO182 polypeptide. Similar articles of manufacture comprising a PRO211, PRO228, PRO538, PRO172 or PRO182 polypeptide as herein defined, or an agonist thereof in an amount that is therapeutically effective for the treatment of tumor are also within the scope of the present invention. Also within the scope of the invention are articles of manufacture comprising a PRO211, PRO228, PRO538, PRO172 or PRO182 polypeptide as herein defined, or an agonist thereof, and a further growth inhibitory agent, cytotoxic agent or chemotherapeutic agent.

### BRIEF DESCRIPTION OF THE DRAWINGS

FIG. 1 shows a nucleotide sequence (SEQ ID NO:1) of a native sequence PRO211 cDNA, wherein SEQ ID NO:1 is a clone designated herein as “DNA32292-1131”.

FIG. 2 shows the amino acid sequence (SEQ ID NO:2) derived from the coding sequence of SEQ ID NO:1 shown in FIG. 1.

FIGS. 3A-B show a nucleotide sequence (SEQ ID NO:6) of a native sequence PRO228 cDNA, wherein SEQ ID NO:6 is a clone designated herein as “DNA33092-1202”.

FIG. 4 shows the amino acid sequence (SEQ ID NO:7) derived from the coding sequence of SEQ ID NO:6 shown in FIGS. 3A-B.

FIG. 5 shows a nucleotide sequence (SEQ ID NO:15) of a native sequence PRO538 cDNA, wherein SEQ ID NO:15 is a clone designated herein as “DNA48613-1268”.

FIG. 6 shows the amino acid sequence (SEQ ID NO:16) derived from the coding sequence of SEQ ID NO:15 shown in FIG. 5.

FIGS. 7A-B show a nucleotide sequence (SEQ ID NO:20) of a native sequence PRO172 cDNA, wherein SEQ ID NO:20 is a clone designated herein as “DNA35916-1161”.

FIG. 8 shows the amino acid sequence (SEQ ID NO:21) derived from the coding sequence of SEQ ID NO:20 shown in FIGS. 7A-B.

FIG. 9 shows a nucleotide sequence (SEQ ID NO:25) of a native sequence PRO182 cDNA, wherein SEQ ID NO:25 is a clone designated herein as “DNA27865-1091 ”.

FIG. 10 shows the amino acid sequence (SEQ ID NO:26) derived from the coding sequence of SEQ ID NO:25 shown in FIG. 9.

### DETAILED DESCRIPTION OF THE INVENTION

The terms “PRO211 ”, “PRO228”, “PRO538”, “PRO172” or “PRO182” polypeptide or protein when used herein encompass native sequence PRO211, PRO228, PRO538, PRO172 and PRO182 variants (which are further defined herein). The PRO211, PRO228, PRO538, PRO172 or PRO182 polypeptide may be isolated from a variety of sources, such as from human tissue types or from another source, or prepared by recombinant and/or synthetic methods.

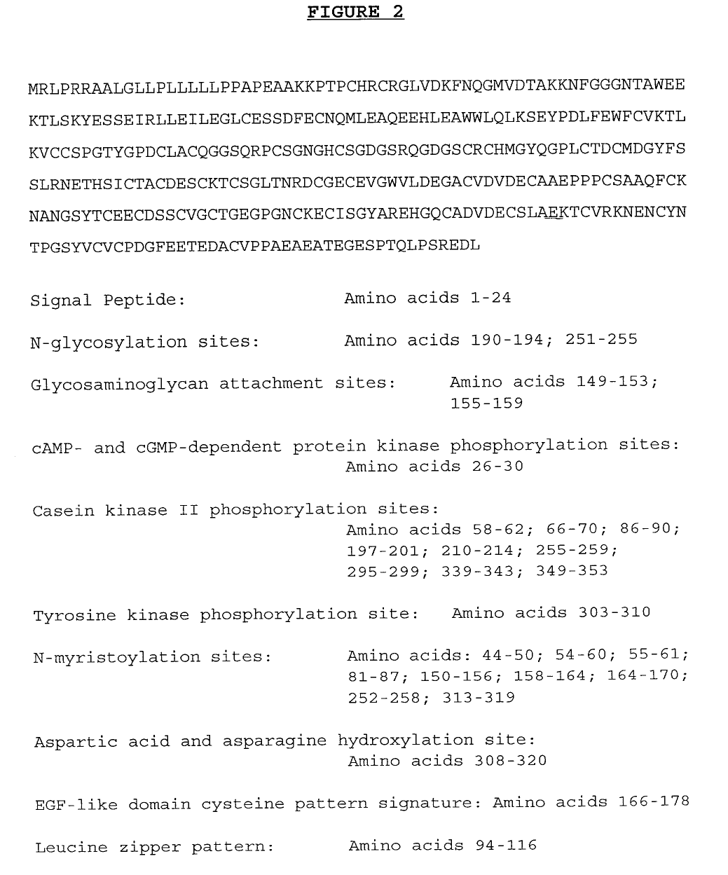

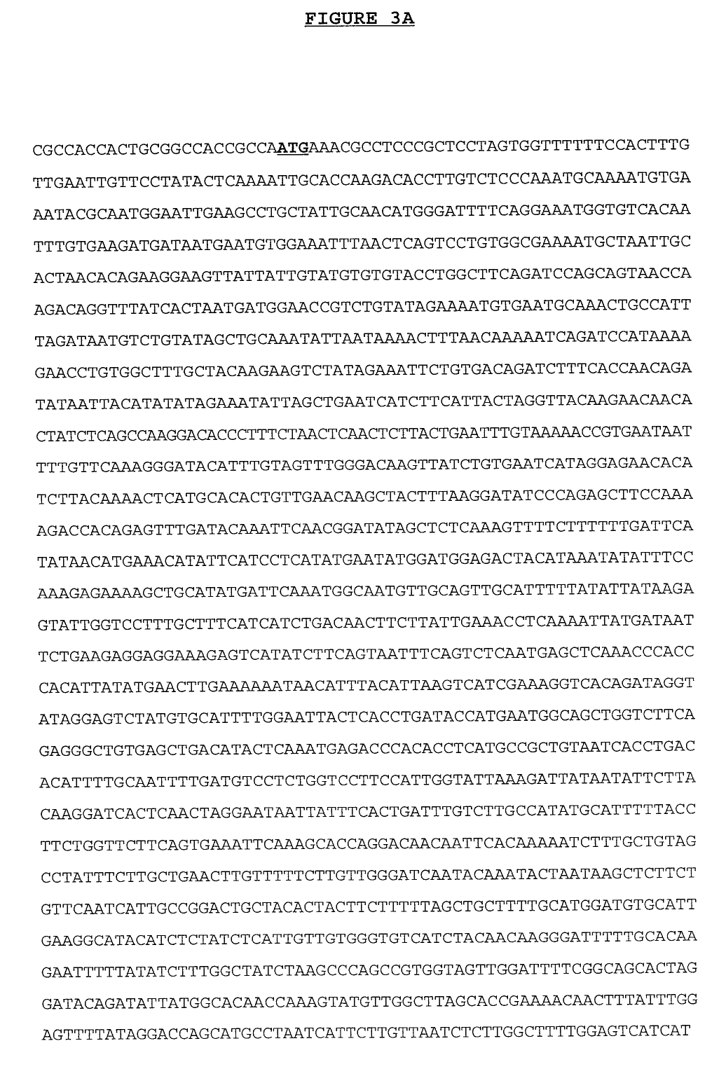

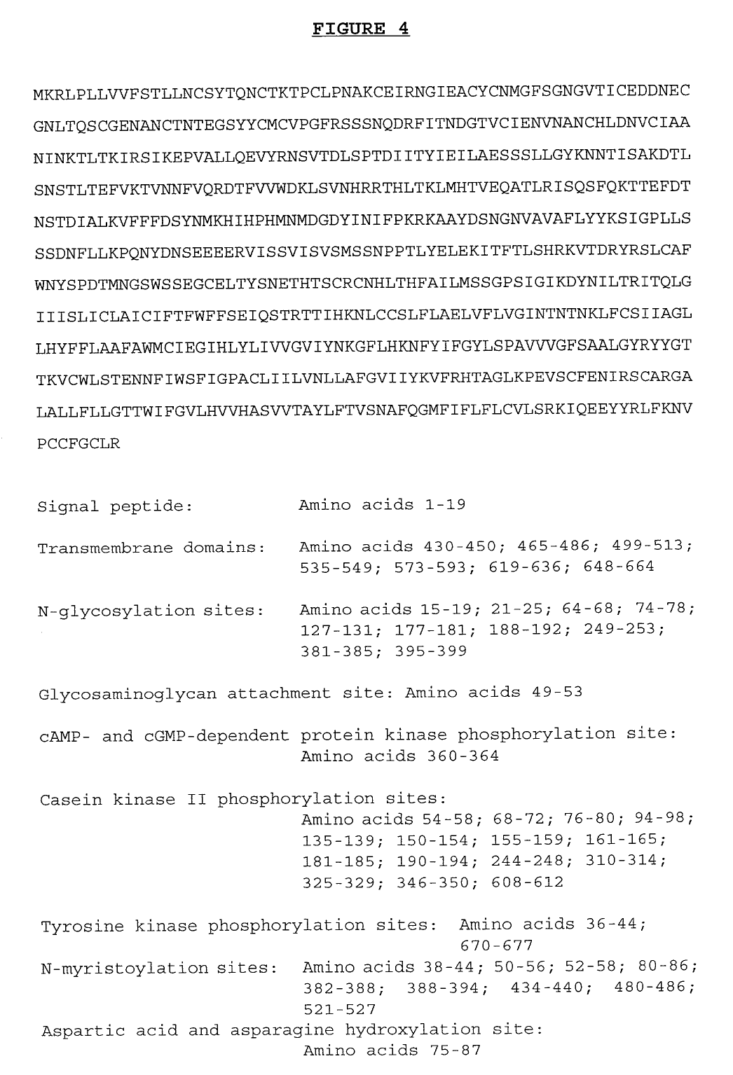

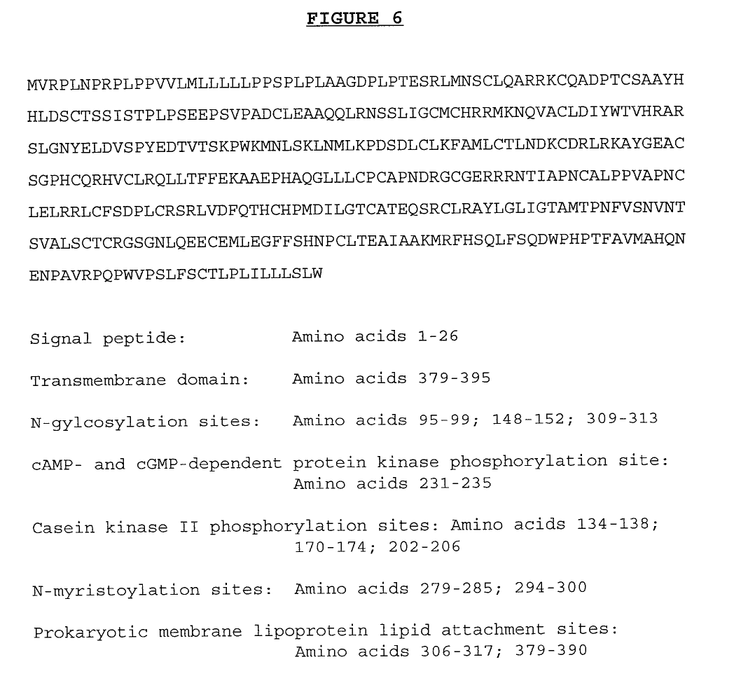

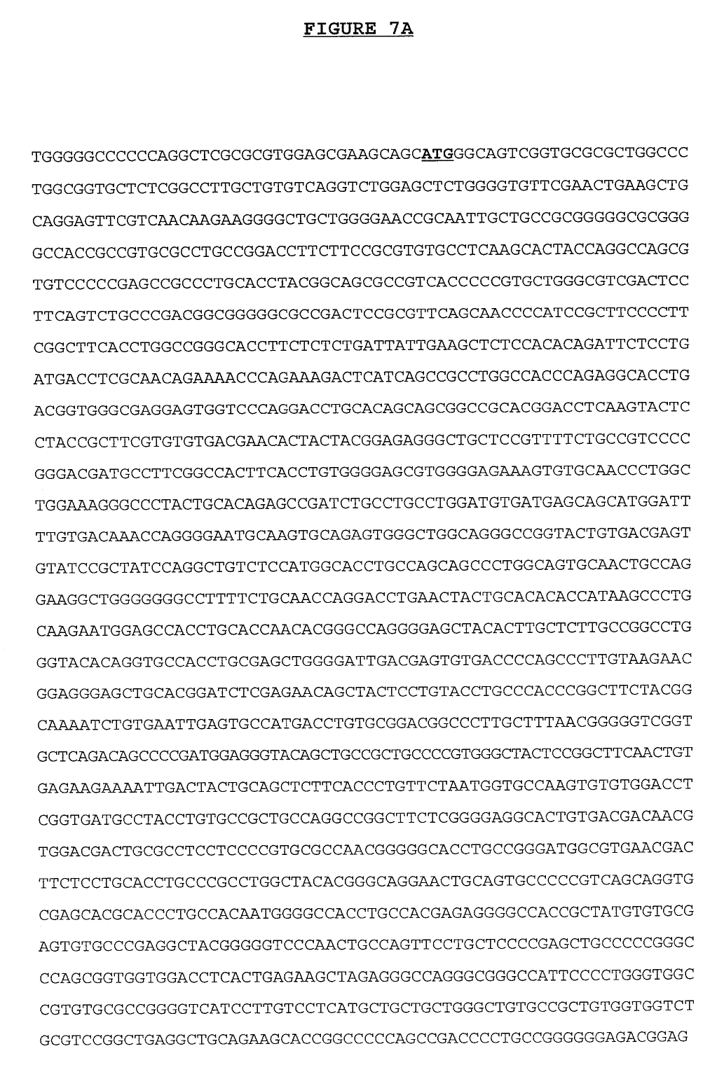

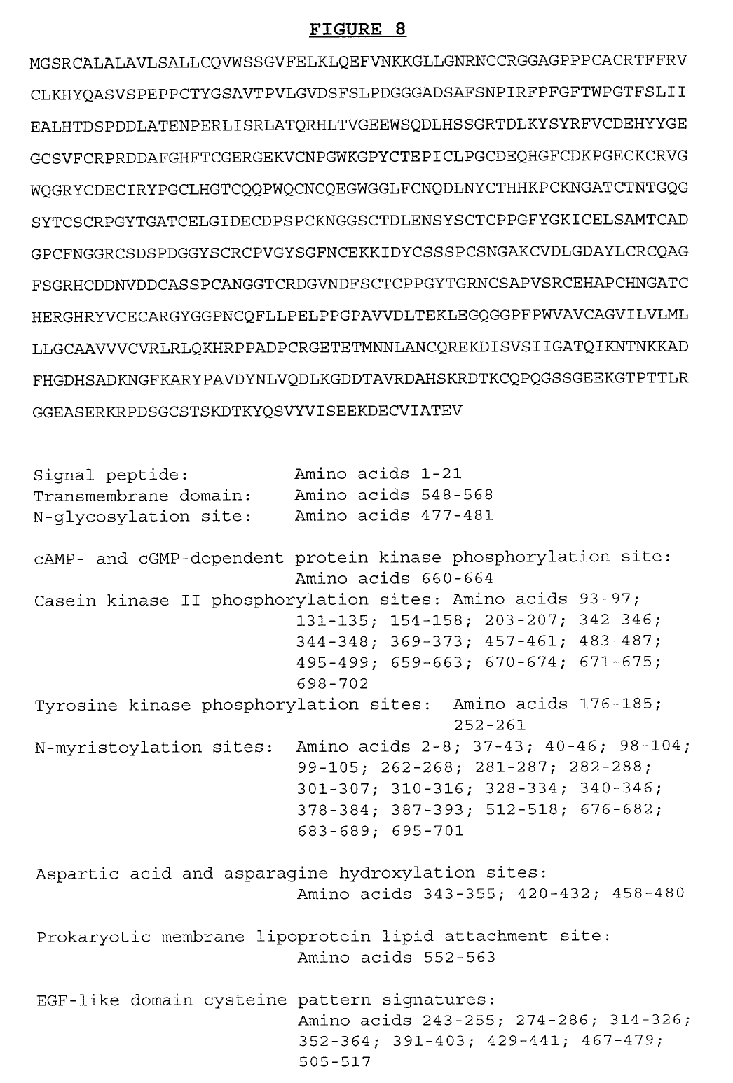

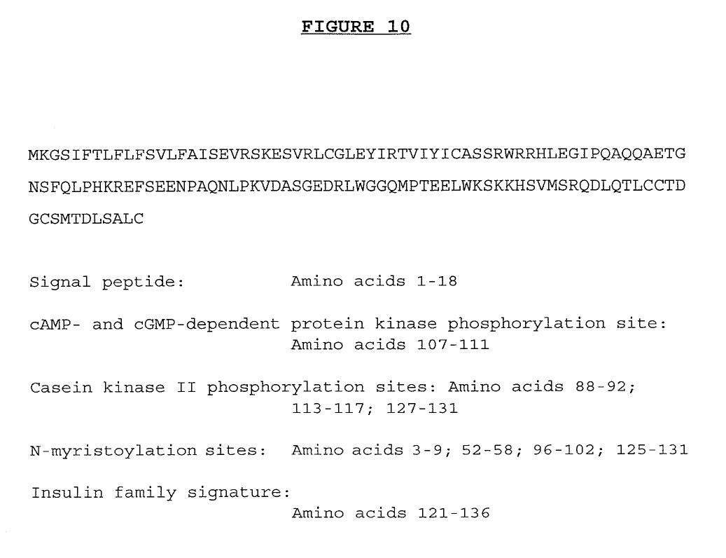

A “native sequence PRO211”, “native sequence PRO228”, “native sequence PRO538”, “native sequence PRO172” or “native sequence PRO182” comprises a polypeptide having the same amino acid sequence as the PRO211, PRO228, PRO538, PRO172 or PRO182 polypeptide as derived from nature. Such native sequence PRO211, PRO228, PRO538, PRO172 or PRO182 polypeptide can be isolated from nature or can be produced by recombinant and/or synthetic means. The term “native sequence” PRO211, PRO228, PRO538, PRO172 or PRO182 specifically encompasses naturally-occurring truncated or secreted forms (e.g., an extracellular domain sequence), naturally-occurring variant forms (e.g., alternatively spliced forms) and naturally-occurring allelic variants of the PRO211, PRO228, PRO538, PRO172 and PRO182 polypeptides. In one embodiment of the invention, the native sequence PRO211, PRO228, PRO538, PRO172 or PRO182 polypeptide is a mature or full-length native sequence PRO211, PRO228, PRO538, PRO172 or PRO182 polypeptide as shown in FIG. 2 (SEQ ID NO:2), FIG. 4 (SEQ ID NO:7), FIG. 6 (SEQ ID NO:16), FIG. 8 (SEQ ID NO:21) or FIG. 10 (SEQ ID NO:26), respectively. Also, while the PRO211, PRO228, PRO538, PRO172 and PRO182 polypeptides disclosed in FIG. 2 (SEQ ID NO:2), FIG. 4 (SEQ ID NO:7), FIG. 6 (SEQ ID NO:16), FIG. 8 (SEQ ID NO:21) and FIG. 10 (SEQ ID NO:26), respectively, are shown to begin with the methionine residue designated therein as amino acid position 1, it is conceivable and possible that another methionine residue located either upstream or downstream from amino acid position 1 in FIG. 2 (SEQ ID NO:2), FIG. 4 (SEQ ID NO:7), FIG. 6 (SEQ ID NO:16), FIG. 8 (SEQ ID NO:21) or FIG. 10 (SEQ ID NO:26), respectively, may be employed as the starting amino acid residue for the PRO211, PRO228, PRO538, PRO172 or PRO182 polypeptide.

The “extracellular domain” or “ECD” of a polypeptide disclosed herein refers to a form of the polypeptide which is essentially free of the transmembrane and cytoplasmic domains. Ordinarily, a polypeptide ECD will have less than about 1% of such transmembrane and/or cytoplasmic domains and preferably, will have less than about 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified and as shown in the appended figures. As such, in one embodiment of the present invention, the extracellular domain of a polypeptide of the present invention comprises amino acids 1 to X of the mature amino acid sequence, wherein X is any amino acid within 5 amino acids on either side of the extracellular domain/transmembrane domain boundary.

The approximate location of the “signal peptides” of the various PRO polypeptides disclosed herein are shown in the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng., 10: 1-6 (1997) and von Heinje et al., Nucl. Acids. Res., 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO211 variant polypeptide” means an active PRO211 polypeptide (other than a native sequence PRO211 polypeptide) as defined below, having at least about 80% amino acid sequence identity with the amino acid sequence of (a) residues 1 or about 25 to 353 of the PRO211 polypeptide shown in FIG. 2 (SEQ ID NO:2), (b) X to 353 of the PRO211 polypeptide shown in FIG. 2 (SEQ ID NO:2), wherein X is any amino acid residue from 20 to 29 of FIG. 2 (SEQ ID NO:2) or (c) another specifically derived fragment of the amino acid sequence shown in FIG. 2 (SEQ ID NO:2).

“PRO228 variant polypeptide” means an active PRO228 polypeptide (other than a native sequence PRO228 polypeptide) as defined below, having at least about 80% amino acid sequence identity with the amino acid sequence of (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7), (c) 1 or about 20 to X of FIG. 4 (SEQ ID NO:7), wherein X is any amino acid from amino acid 425 to amino acid 434 of FIG. 4 (SEQ ID NO:7) or (d) another specifically derived fragment of the amino acid sequence shown in FIG. 4 (SEQ ID NO:7).

“PRO538 variant polypeptide” means an active PRO538 polypeptide (other than a native sequence PRO538 polypeptide) as defined below, having at least about 80% amino acid sequence identity with the amino acid sequence of (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16), (c) 1 or about 27 to X of FIG. 6 (SEQ ID NO:16), wherein X is any amino acid from amino acid 374 to amino acid 383 of FIG. 6 (SEQ ID NO:16) or (d) another specifically derived fragment of the amino acid sequence shown in FIG. 6 (SEQ ID NO:16).

“PRO172 variant polypeptide” means an active PRO172 polypeptide (other than a native sequence PRO172 polypeptide) as defined below, having at least about 80% amino acid sequence identity with the amino acid sequence of (a) residues 1 or about 22 to 723 of the PRO172 polypeptide shown in FIG. 8 (SEQ ID NO:21), (b) X to 723 of the PRO172 polypeptide shown in FIG. 8 (SEQ ID NO:21), wherein X is any amino acid residue from 17 to 26 of FIG. 8 (SEQ ID NO:21), (c) 1 or about 22 to X of FIG. 8 (SEQ ID NO:21), wherein X is any amino acid from amino acid 543 to amino acid 552 of FIG. 8 (SEQ ID NO:21) or (d) another specifically derived fragment of the amino acid sequence shown in FIG. 8 (SEQ ID NO:21).

“PRO182 variant polypeptide” means an active PRO182 polypeptide (other than a native sequence PRO182 polypeptide) as defined below, having at least about 80% amino acid sequence identity with the amino acid sequence of (a) residues 1 or about 19 to 135 of the PRO182 polypeptide shown in FIG. 10 (SEQ ID NO:26), (b) X to 135 of the PRO182 polypeptide shown in FIG. 10 (SEQ ID NO:26), wherein X is any amino acid residue from 14 to 23 of FIG. 10 (SEQ ID NO:26) or (c) another specifically derived fragment of the amino acid sequence shown in FIG. 10 (SEQ ID NO:26).

Such PRO211, PRO228, PRO538, PRO172 and PRO182 variants include, for instance, PRO211, PRO228, PRO538, PRO172 and PRO182 polypeptides wherein one or more amino acid residues are added, or deleted, at the N- or C-terminus, as well as within one or more internal domains of the native sequence.

Ordinarily, a PRO211 variant will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues 1 or about 25 to 353 of the PRO21 polypeptide shown in FIG. 2 (SEQ ID NO:2), (b) X to 353 of the PRO211 polypeptide shown in FIG. 2 (SEQ ID NO:2), wherein X is any amino acid residue from 20 to 29 of FIG. 2 (SEQ ID NO:2) or (c) another specifically derived fragment of the amino acid sequence shown in FIG. 2 (SEQ ID NO:2).

Ordinarily, a PRO228 variant will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7), (c) 1 or about 20 to X of FIG. 4 (SEQ ID NO:7), wherein X is any amino acid from amino acid 425 to amino acid 434 of FIG. 4 (SEQ ID NO:7) or (d) another specifically derived fragment of the amino acid sequence shown in FIG. 4 (SEQ ID NO:7).

Ordinarily, a PRO538 variant will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16), (c) 1 or about 27 to X of FIG. 6 (SEQ ID NO:16), wherein X is any amino acid from amino acid 374 to amino acid 383 of FIG. 6 (SEQ ID NO:16) or (d) another specifically derived fragment of the amino acid sequence shown in FIG. 6 (SEQ ID NO:16).

Ordinarily, a PRO172 variant will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% am ino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues 1 or about 22 to 723 of the PRO172 polypeptide shown in FIG. 8 (SEQ ID NO:21), (b) X to 723 of the PRO172 polypeptide shown in FIG. 8 (SEQ ID NO:21), wherein X is any amino acid residue from 17 to 26 of FIG. 8 (SEQ ID NO:21), (c) 1 or about 22 to X of FIG. 8 (SEQ ID NO:21), wherein X is any amino acid from amino acid 543 to amino acid 552 of FIG. 8 (SEQ ID NO:21) or (d) another specifically derived fragment of the amino acid sequence shown in FIG. 8 (SEQ ID NO:21).

Ordinarily, a PRO182 variant will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with (a) residues 1 or about 19 to 135 of the PRO182 polypeptide shown in FIG. 10 (SEQ ID NO:26), (b) X to 135 of the PRO182 polypeptide shown in FIG. 10 (SEQ ID NO:26), wherein X is any amino acid residue from 14 to 23 of FIG. 10 (SEQ ID NO:26) or (c) another specifically derived fragment of the amino acid sequence shown in FIG. 10 (SEQ ID NO:26).

Ordinarily, PRO211, PRO228, PRO538, PRO172 and PRO182 variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

As shown below, Table 1 provides the complete source code for the ALIGN-2 sequence comparison computer program. This source code may be routinely compiled for use on a UNIX operating system to provide the ALIGN-2 sequence comparison computer program.

In addition, Tables 2A-2B show hypothetical exemplifications for using the below described method to determine % amino acid sequence identity (Tables 2A-2B) and % nucleic acid sequence identity (Tables 2C-2D) using the ALIGN-2 sequence comparison computer program, wherein “PRO” represents the amino acid sequence of a hypothetical PEACH polypeptide of interest, “Comparison Protein” represents the amino acid sequence of a polypeptide against which the “PRO” polypeptide of interest is being compared, “PRO-DNA” represents a hypothetical PROXXX- or PROXXX-encoding nucleic acid sequence of interest, “Comparison DNA” represents the nucleotide sequence of a nucleic acid molecule against which the “PRO-DNA” nucleic acid molecule of interest is being compared, “X”, “Y”, and “Z” each represent different hypothetical amino acid residues and “N”, “L” and “V” each represent different hypothetical nucleotides.

[image]

[image]

[image]

[image]

[image]

[image]

[image]

[image]

[image]

[image]

[image]

[image]

[image]

[image]

[image]

[image]

[image]

TABLE 2A

| | | |
|---|---|---|
| PRO | XXXXXXXXXXXXXXX | (Length = |
| | | 15 amino acids) |
| Comparison Protein | XXXXXYYYYYYY | (Length = |
| | | 12 amino acids) |

% amino acid sequence identity =
(the number of identically matching amino acid residues between the two
polypeptide sequences as determined by ALIGN-2) divided by (the total
number of amino acid residues of the PRO polypeptide) = 5 divided by
15 = 33.3%

TABLE 2B

| | | |
|---|---|---|
| PRO | XXXXXXXXXX | (Length = 10 |
| | | amino acids) |
| Comparison Protein | XXXXXYYYYYYZZYZ | (Length = 15 |
| | | amino acids) |

% amino acid sequence identity =
(the number of identically matching amino acid residues between the two
polypeptide sequences as determined by ALIGN-2) divided by (the total
number of amino acid residues of the PRO polypeptide) = 5 divided by
10 = 50%

TABLE 2C

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity =
(the number of identically matching nucleotides between the two nucleic
acid sequences as determined by ALIGN-2) divided by (the total number
of nucleotides of the PRO-DNA nucleic acid sequence) = 6 divided by
14 = 42.9%

TABLE 2D

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity =
(the number of identically matching nucleotides between the two nucleic
acid sequences as determined by ALIGN-2) divided by (the total number
of nucleotides of the PRO-DNA nucleic acid sequence) = 4 divided by
12 = 33.3%

“Percent (%) amino acid sequence identity” with respect to the PRO211, PRO228, PRO538, PRO172 and PRO182 polypeptide sequences identified herein is defined as the percentage of amino acid residues in a candidate sequence that are identical with the amino acid residues in a PRO211, PRO228, PRO538, PRO172 or PRO182 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table I. The ALIGN-2 sequence comparison computer program was authored by Genentech, Inc., and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

### 100 Times the Fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Tables 2A-2B demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

### 100 Times the Fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In addition, % amino acid sequence identity may also be determined using the WU-BLAST-2 computer program (Altschul et al., Methods in Enzymology, 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % amino acid sequence identity value is determined by dividing (a) the number of matching identical amino acids residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

“PRO211 variant polynucleotide” or “PRO211 variant nucleic acid sequence” means a nucleic acid molecule which encodes an active PRO211 polypeptide as defined below and which has at least about 80% nucleic acid sequence identity with either (a) a nucleic acid sequence which encodes residues 1 or about 25 to 353 of the PRO211 polypeptide shown in FIG. 2 (SEQ ID NO:2), (b) a nucleic acid sequence which encodes amino acids X to 353 of the PRO211 polypeptide shown in FIG. 2 (SEQ ID NO:2), wherein X is any amino acid residue from 20 to 29 of FIG. 2 (SEQ ID NO:2) or (c) a nucleic acid sequence which encodes another specifically derived fragment of the amino acid sequence shown in FIG. 2 (SEQ ID NO:2). Ordinarily, a PRO211 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues 1 or about 25 to 353 of the PRO211 polypeptide shown in FIG. 2 (SEQ ID NO:2), (b) a nucleic acid sequence which encodes amino acids X to 353 of the PRO211 polypeptide shown in FIG. 2 (SEQ ID NO:2), wherein X is any amino acid residue from 20 to 29 of FIG. 2 (SEQ ID NO:2) or (c) a nucleic acid sequence which encodes another specifically derived fragment of the amino acid sequence shown in FIG. 2 (SEQ ID NO:2). PRO211 polynucleotide variants do not encompass the native PRO211 nucleotide sequence.

“PRO228 variant polynucleotide” or “PRO228 variant nucleic acid sequence” means a nucleic acid molecule which encodes an active PRO228 polypeptide as defined below and which has at least about 80% nucleic acid sequence identity with either (a) a nucleic acid sequence which encodes residues 1 or about 20 to 690 of the PRO228 polypeptide shown in FIG. 4 (SEQ ID NO:7), (b) a nucleic acid sequence which encodes amino acids X to 690 of the PRO228 polypeptide shown in FIG. 4 (SEQ ID NO:7), wherein X is any amino acid residue from 15 to 24 of FIG. 4 (SEQ ID NO:7), (c) a nucleic acid sequence which encodes amino acids 1 or about 20 to X of FIG. 4 (SEQ ID NO:7), wherein X is any amino acid from amino acid 425 to amino acid 434 of FIG. 4 (SEQ ID NO:7) or (d) a nucleic acid sequence which encodes another specifically derived fragment of the amino acid sequence shown in FIG. 4 (SEQ ID NO:7). Ordinarily, a PRO228 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues 1 or about 20 to 690 of the PRO228 polypeptide shown in FIG. 4 (SEQ ID NO:7), (b) a nucleic acid sequence which encodes amino acids X to 690 of the PRO228 polypeptide shown in FIG. 4 (SEQ ID NO:7), wherein X is any amino acid residue from 15 to 24 of FIG. 4 (SEQ ID NO:7), (c) a nucleic acid sequence which encodes amino acids 1 or about 20 to X of FIG. 4 (SEQ ID NO:7), wherein X is any amino acid from amino acid 425 to amino acid 434 of FIG. 4 (SEQ ID NO:7) or (d) a nucleic acid sequence which encodes another specifically derived fragment of the amino acid sequence shown in FIG. 4 (SEQ ID NO:7). PRO228 polynucleotide variants do not encompass the native PRO228 nucleotide sequence.

“PRO538 variant polynucleotide” or “PRO538 variant nucleic acid sequence” means a nucleic acid molecule which encodes an active PRO538 polypeptide as defined below and which has at least about 80% nucleic acid sequence identity with either (a) a nucleic acid sequence which encodes residues 1 or about 27 to 400 of the PRO538 polypeptide shown in FIG. 6 (SEQ ID NO:16), (b) a nucleic acid sequence which encodes amino acids X to 400 of the PRO538 polypeptide shown in FIG. 6 (SEQ ID NO:16), wherein X is any amino acid residue from 22 to 31 of FIG. 6 (SEQ ID NO:16), (c) a nucleic acid sequence which encodes amino acids 1 or about 27 to X of FIG. 6 (SEQ ID NO:16), wherein X is any amino acid from amino acid 374 to amino acid 383 of FIG. 6 (SEQ ID NO:16) or (d) a nucleic acid sequence which encodes another specifically derived fragment of the amino acid sequence shown in FIG. 6 (SEQ ID NO:16). Ordinarily, a PRO538 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues 1 or about 27 to 400 of the PRO538 polypeptide shown in FIG. 6 (SEQ ID NO:16), (b) a nucleic acid sequence which encodes amino acids X to 400 of the PRO538 polypeptide shown in FIG. 6 (SEQ ID NO:16), wherein X is any amino acid residue from 22 to 31 of FIG. 6 (SEQ ID NO:16), (c) a nucleic acid sequence which encodes amino acids 1 or about 27 to X of FIG. 6 (SEQ ID NO:16), wherein X is any amino acid from amino acid 374 to amino acid 383 of FIG. 6 (SEQ ID NO:16) or (d) a nucleic acid sequence which encodes another specifically derived fragment of the amino acid sequence shown in FIG. 6 (SEQ ID NO:16). PRO538 polynucleotide variants do not encompass the native PRO538 nucleotide sequence.

“PRO172 variant polynucleotide” or “PRO172 variant nucleic acid sequence” means a nucleic acid molecule which encodes an active PRO172 polypeptide as defined below and which has at least about 80% nucleic acid sequence identity with either (a) a nucleic acid sequence which encodes residues 1 or about 22 to 723 of the PRO172 polypeptide shown in FIG. 8 (SEQ ID NO:21), (b) a nucleic acid sequence which encodes amino acids X to 723 of the PRO172 polypeptide shown in FIG. 8 (SEQ ID NO:21), wherein X is any amino acid residue from 17 to 26 of FIG. 8 (SEQ ID NO:21), (c) a nucleic acid sequence which encodes amino acids 1 or about 22 to X of FIG. 8 (SEQ ID NO:21), wherein X is any amino acid from amino acid 543 to amino acid 552 of FIG. 8 (SEQ ID NO:21) or (d) a nucleic acid sequence which encodes another specifically derived fragment of the amino acid sequence shown in FIG. 8 (SEQ ID NO:21). Ordinarily, a PRO172 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues 1 or about 22 to 723 of the PRO173 polypeptide shown in FIG. 8 (SEQ ID NO:21), (b) a nucleic acid sequence which encodes amino acids X to 723 of the PRO172 polypeptide shown in FIG. 8 (SEQ ID NO:21), wherein X is any amino acid residue from 17 to 26 of FIG. 8 (SEQ ID NO:21), (c) a nucleic acid sequence which encodes amino acids 1 or about 22 to X of FIG. 8 (SEQ ID NO:21), wherein X is any amino acid from amino acid 543 to amino acid 552 of FIG. 8 (SEQ ID NO:21) or (d) a nucleic acid sequence which encodes another specifically derived fragment of the amino acid sequence shown in FIG. 8 (SEQ ID NO:21). PRO172 polynucleotide variants do not encompass the native PRO172 nucleotide sequence.

“PRO182 variant polynucleotide” or “PRO182 variant nucleic acid sequence” means a nucleic acid molecule which encodes an active PRO182 polypeptide as defined below and which has at least about 80% nucleic acid sequence identity with either (a) a nucleic acid sequence which encodes residues 1 or about 19 to 135 of the PRO182 polypeptide shown in FIG. 10 (SEQ ID NO:26), (b) a nucleic acid sequence which encodes amino acids X to 135 of the PRO182 polypeptide shown in FIG. 10 (SEQ ID NO:26), wherein X is any amino acid residue from 14 to 23 of FIG. 10 (SEQ ID NO:26) or (c) a nucleic acid sequence which encodes another specifically derived fragment of the amino acid sequence shown in FIG. 10 (SEQ ID NO:26). Ordinarily, a PRO182 variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes residues 1 or about 19 to 135 of the PRO182 polypeptide shown in FIG. 10 (SEQ ID NO:26), (b) a nucleic acid sequence which encodes amino acids X to 135 of the PRO182 polypeptide shown in FIG. 10 (SEQ ID NO:26), wherein X is any amino acid residue from 14 to 23 of FIG. 10 (SEQ ID NO:26) or (c) a nucleic acid sequence which encodes another specifically derived fragment of the amino acid sequence shown in FIG. 10 (SEQ ID NO:26). PRO182 polynucleotide variants do not encompass the native PRO182 nucleotide sequence.

Ordinarily, PRO211, PRO228, PRO538, PRO172 and PRO182 variant polynucleotides are at least about 30 nucleotides in length, often at least about 60 nucleotides in length, more often at least about 90 nucleotides in length, more often at least about 120 nucleotides in length, more often at least about 150 nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to the PRO211, PRO228, PRO538, PRO172 and PRO182 polypeptide-encoding nucleic acid sequences identified herein is defined as the percentage of nucleotides in a candidate sequence that are identical with the nucleotides in a PRO211, PRO228, PRO538, PRO172 or PRO182 polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc., and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

### 100 Times the Fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 2C-2D demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-passe-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

### 100 Times the Fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In addition, % nucleic acid sequence identity values may also be generated using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

In other embodiments, PRO211, PRO228, PRO538, PRO172 and PRO182 variant polynucleotides are nucleic acid molecules that encode an active PRO211, PRO228, PRO538, PRO172 or PRO182 polypeptide, respectively, and which are capable of hybridizing, preferably under stringent hybridization and wash conditions, to nucleotide sequences encoding the full-length PRO211 polypeptide shown in FIG. 2 (SEQ ID NO:2), to nucleotide sequences encoding the full-length PRO228 polypeptide shown in FIG. 4 (SEQ ID NO:7), to nucleotide sequences encoding the full-length PRO538 polypeptide shown in FIG. 6 (SEQ ID NO:16), to nucleotide sequences encoding the full-length PRO172 polypeptide shown in FIG. 8 (SEQ ID NO:21), to nucleotide sequences encoding the full-length PRO182 polypeptide shown in FIG. 10 (SEQ ID NO:26), respectively. PRO211, PRO228, PRO538, PRO172 and PRO182 variant polypeptides may be those that are encoded by a PRO211, PRO228, PRO538, PRO172 or PRO182 variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 3 below) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows:

### 100 Times the Fraction X/Y

where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO211, PRO228, PRO538, PRO172 or PRO182 natural environment will not be present. Ordinarily, however, isolated polypeptide will be prepared by at least one purification step.

An “isolated” nucleic acid molecule encoding a PRO211, PRO228, PRO538, PRO172 or PRO182 polypeptide or an “isolated” nucleic acid molecule encoding an anti-PRO211, anti-PRO228, anti-PRO538, anti-PRO172 or anti-PRO182 antibody is a nucleic acid molecule that is identified and separated from at least one contaminant nucleic acid molecule with which it is ordinarily associated in the natural source of the PRO211-, PRO228-, PRO538-, PRO172- or PRO182-encoding nucleic acid or the anti-PRO211-, anti-PRO228-, anti-PRO538-, anti-PRO172- or anti-PRO182-encoding nucleic acid. Preferably, the isolated nucleic acid is free of association with all components with which it is naturally associated. An isolated PRO211-, PRO228-, PRO538-, PRO172- or PRO182-encoding nucleic acid molecule or an isolated anti-PRO211-, anti-PRO228-, anti-PRO538-, anti-PRO172- or anti-PRO182-encoding nucleic acid molecule is other than in the form or setting in which it is found in nature. Isolated nucleic acid molecules therefore are distinguished from the PRO211-, PRO228-, PRO538, -PRO172- or PRO182-encoding nucleic acid molecule or from the anti-PRO211-, anti-PRO228-, anti-PRO538-, anti-PRO172- or anti-PRO182-encoding nucleic acid molecule as it exists in natural cells. However, an isolated nucleic acid molecule encoding a PRO211, PRO228, PRO538, PRO172 or PRO182 polypeptide or an isolated nucleic acid molecule encoding an anti-PRO211, anti-PRO228, anti-PRO538, anti-PRO172 or anti-PRO182 antibody includes PRO211-, PRO228-, PRO538-, PRO172- or PRO182-nucleic acid molecules or anti-PRO211-, anti-PRO228-, anti-PRO538-, anti-PRO172- or anti-PRO182-nucleic acid molecules contained in cells that ordinarily express PRO211, PRO228, PRO538, PRO172 or PRO182 polypeptides or anti-PRO211, anti-PRO228, anti-PRO538, anti-PRO172 or anti-PRO182 antibodies where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO211, anti-PRO228, anti-PRO538, anti-PRO172 and anti-PRO182 monoclonal antibodies (including agonist antibodies), anti-PRO211, anti-PRO228, anti-PRO538, anti-PRO172 and anti-PRO182 antibody compositions with polyepitopic specificity, single chain anti-PRO211, anti-PRO228, anti-PRO538, anti-PRO172 and anti-PRO182 antibodies, and fragments of anti-PRO211, anti-PRO228, anti-PRO538, anti-PRO172 and anti-PRO182 antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0. 1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5× SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1× SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO211, PRO228, PRO538, PRO172 or PRO182 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-I and IgA-2), IgE, IgD or IgM.

“Active” or “activity” for the purposes herein refers to form(s) of PRO211, PRO228, PRO538, PRO172 or PRO182 which retain a biological and/or an immunological activity of native or naturally-occurring PRO211, PRO228, PRO538, PRO172 or PRO182, wherein “biological” activity refers to a biological function (either inhibitory or stimulatory) caused by a native or naturally-occurring PRO211, PRO228, PRO538, PRO172 or PRO182 other than the ability to induce the production of an antibody against an antigenic epitope possessed by a native or naturally-occurring PRO211, PRO228, PRO538, PRO172 or PRO182 and an “immunological” activity refers to the ability to induce the production of an antibody against an antigenic epitope possessed by a native or naturally-occurring PRO211, PRO228, PRO538, PRO172 or PRO182.

“Biological activity” in the context of an antibody or another agonist that can be identified by the screening assays disclosed herein (e.g., an organic or inorganic small molecule, peptide, etc.) is used to refer to the ability of such molecules to invoke one or more of the effects listed herein in connection with the definition of a “therapeutically effective amount.” In a specific embodiment, “biological activity” is the ability to inhibit neoplastic cell growth or proliferation. A preferred biological activity is inhibition, including slowing or complete stopping, of the growth of a target tumor (e.g., cancer) cell. Another preferred biological activity is cytotoxic activity resulting in the death of the target tumor (e.g., cancer) cell. Yet another preferred biological activity is the induction of apoptosis of a target tumor (e.g., cancer) cell.

The phrase “immunological activity” means immunological cross-reactivity with at least one epitope of a PRO211, PRO228, PRO538, PRO172 or PRO182 polypeptide.

“Immunological cross-reactivity” as used herein means that the candidate polypeptide is capable of competitively inhibiting the qualitative biological activity of a PRO211, PRO228, PRO538, PRO172 or PRO182 polypeptide having this activity with polyclonal antisera raised against the known active PRO211, PRO228, PRO538, PRO172 or PRO182 polypeptide. Such antisera are prepared in conventional fashion by injecting goats or rabbits, for example, subcutaneously with the known active analogue in complete Freund's adjuvant, followed by booster intraperitoneal or subcutaneous injection in incomplete Freunds. The immunological cross-reactivity preferably is “specific”, which means that the binding affinity of the immunologically cross-reactive molecule (e.g., antibody) identified, to the corresponding PRO211, PRO228, PRO538, PRO172 or PRO182 polypeptide is significantly higher (preferably at least about 2-times, more preferably at least about 4-times, even more preferably at least about 6-times, most preferably at least about 8-times higher) than the binding affinity of that molecule to any other known native polypeptide.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, endometrial carcinoma, salivary gland carcinoma, kidney cancer, vulval cancer, thyroid cancer, hepatic carcinoma and various types of head and neck cancer.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

An “effective amount” of a polypeptide disclosed herein or an agonist thereof, in reference to inhibition of neoplastic cell growth, is an amount capable of inhibiting, to some extent, the growth of target cells. The term includes an amount capable of invoking a growth inhibitory, cytostatic and/or cytotoxic effect and/or apoptosis of the target cells. An “effective amount” of a PRO211, PRO228, PRO538, PRO172 or PRO182 polypeptide or an agonist thereof for purposes of inhibiting neoplastic cell growth may be determined empirically and in a routine manner.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a PRO211, PRO228, PRO538, PRO172 or PRO182 polypeptide or an agonist thereof for purposes of treatment of tumor may be determined empirically and in a routine manner.

A “growth inhibitory amount” of a PRO211, PRO228, PRO538, PRO172 or PRO182 polypeptide or an agonist thereof is an amount capable of inhibiting the growth of a cell, especially tumor, e.g., cancer cell, either in vitro or in vivo. A “growth inhibitory amount” of a PRO211, PRO228, PRO538, PRO172 or PRO182 polypeptide or an agonist thereof for purposes of inhibiting neoplastic cell growth may be determined empirically and in a routine manner.

A “cytotoxic amount” of a PRO211, PRO228, PRO538, PRO172 or PRO182 polypeptide or an agonist thereof is an amount capable of causing the destruction of a cell, especially tumor, e.g., cancer cell, either in vitro or in vivo. A “cytotoxic amount” of a PRO211, PRO228, PRO538, PRO172 or PRO182 polypeptide or an agonist thereof for purposes of inhibiting neoplastic cell growth may be determined empirically and in a routine manner.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of tumor, e.g., cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, Rnace), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see, U.S. Pat. No. 4,675,187), melphalan and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially tumor, e.g., cancer cell, either in vitro or in vivo. Thus, the growth inhibitory agent is one which significantly reduces the percentage of the target cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al., (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g. Wilman, “Prodrugs in Cancer Chemotherapy”, Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, glycosylated prodrugs or optionally substituted phenylacetamide-containing prodrugs, 5-fluorocytosine and other 5-fluorouridine prodrugs which can be derivatized into a prodrug form for use in this invention include, but are not limited to, those chemotherapeutic agents described above.

The term “agonist” is used in the broadest sense and includes any molecule that mimics a biological activity of a native PRO211, PRO228, PRO538, PRO172 or PRO182 polypeptide disclosed herein. Suitable agonist molecules specifically include agonist antibodies or antibody fragments, fragments or amino acid sequence variants of native PRO211, PRO228, PRO538, PRO172 or PRO182 polypeptides, peptides, small organic molecules, etc. Methods for identifying agonists of a PRO211, PRO228, PRO538, PRO172 or PRO182 polypeptide may comprise contacting a tumor cell with a candidate agonist and measuring the inhibition of tumor cell growth.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a βsheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see, Kabat et al., NIH Publ. No.91-3242, Vol. I, pages 647-669 (1991)). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institute of Health, Bethesda, Md. [1991]) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Clothia and Lesk, J. Mol. Biol., 196:901-917 [1987]). “Framework” or “FR” residues are those variable domain residues other than the hypervariable region residues as herein defined.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 [1991] and Marks et al., J. Mol. Biol., 222:581-597(1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see, Jones et al., Nature, 321:522-525 (1986); Reichmann et al., Nature, 332:323-329 [1988]; and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see, Pluckthun in The Pharmacology of Monoclonal Antibodies Vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO211, PRO228, PRO538, PRO1172 or PRO1182 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

II. Compositions and Methods of the Invention

A. Full-length PRO211, PRO228, PRO538, PRO172 and PRO182 Polypeptides

The present invention provides newly identified and isolated nucleotide sequences encoding polypeptides referred to in the present application as PRO211, PRO228, PRO538, PRO172 and PRO182. In particular, cDNAs encoding PRO211, PRO228, PRO538, PRO172 and PRO182 polypeptides have been identified and isolated, as disclosed in further detail in the Examples below.

As disclosed in the Examples below, cDNA clones encoding PRO211, PRO228, PRO538, PRO172 and PRO182 polypeptides have been deposited with the ATCC. The actual nucleotide sequences of the clones can readily be determined by the skilled artisan by sequencing of the deposited clones using routine methods in the art. The predicted amino acid sequences can be determined from the nucleotide sequences using routine skill. For the PRO211, PRO228, PRO538, PRO172 and PRO182 polypeptides and encoding nucleic acids described herein, Applicants have identified what is believed to be the reading frame best identifiable with the sequence information available at the time.

B. PRO211, PRO228, PRO538 PRO172 and PRO182 Variants

In addition to the full-length native sequence PRO211, PRO228, PRO538, PRO172 and PRO182 polypeptides described herein, it is contemplated that PRO211, PRO228, PRO538, PRO172 and PRO182 variants can be prepared. PRO211, PRO228, PRO538, PRO172 and PRO182 variants can be prepared by introducing appropriate nucleotide changes into the PRO211, PRO228, PRO538, PRO172 or PRO182 DNA, and/or by synthesis of the desired PRO211, PRO228, PRO538, PRO172 or PRO182 polypeptide. Those skilled in the art will appreciate that amino acid changes may alter post-translational processes of the PRO211, PRO228, PRO538, PRO172 or PRO182 polypeptide, such as changing the number or position of glycosylation sites or altering the membrane anchoring characteristics.

Variations in the native full-length sequence PRO211, PRO228, PRO538, PRO172 or PRO182 or in various domains of the PRO211, PRO228, PRO538, PRO172 or PRO182 described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO211, PRO228, PRO538, PRO172 or PRO182 that results in a change in the amino acid sequence of the PRO211, PRO228, PRO538, PRO172 or PRO182 as compared with the native sequence PRO211, PRO228, PRO538, PRO172 or PRO182. Optionally the variation is by substitution of at least one amino acid with any other amino acid in one or more of the domains of the PRO211, PRO228, PRO538, PRO172 or PRO182. Guidance in determining which amino acid residue may be inserted, substituted or deleted without adversely affecting the desired activity may be found by comparing the sequence of the PRO211, PRO228, PRO538, PRO172 or PRO182 with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO211, PRO228, PRO538, PRO172 and PRO182 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO211, PRO228, PRO538, PRO172 or PRO182 polypeptide.

PRO211, PRO228, PRO538, PRO172 and PRO182 fragments may be prepared by any of a number of conventional techniques. Desired peptide fragments may be chemically synthesized. An alternative approach involves generating PRO211, PRO228, PRO538, PRO172 and PRO182 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO211, PRO228, PRO538, PRO172 and PRO182 polypeptide fragments share at least one biological and/or immunological activity with the native PRO211, PRO228, PRO538, PRO172 or PRO182 polypeptide shown in FIG. 2 (SEQ ID NO:2), FIG. 4 (SEQ ID NO:7), FIG. 6 (SEQ ID NO:16), FIG. 8 (SEQ ID NO:21) and FIG. 10 (SEQ ID NO:26), respectively.

In particular embodiments, conservative substitutions of interest are shown in Table 3 under the heading of preferred substitutions. If such substitutions result in a change in biological activity, then more substantial changes, denominated exemplary substitutions in Table 3, or as further described below in reference to amino acid classes, are introduced and the products screened.

TABLE 3

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO211, PRO228, PRO538, PRO172 or PRO182 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315(1985)], restriction selection mutagenesis [Wells et al., Philos. Trans. R. Soc. London SerA, 317:415 (1986)] or other known techniques can be performed on the cloned DNA to produce the PRO211, PRO228, PRO538, PRO172 or PRO182 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO211, PRO228, PRO538, PRO172 and PRO182

Covalent modifications of PRO211, PRO228, PRO538, PRO172 and PRO182 are included within the scope of this invention. One type of covalent modification includes reacting targeted amino acid residues of a PRO211, PRO228, PRO538, PRO172 or PRO182 polypeptide with an organic derivatizing agent that is capable of reacting with selected side chains or the N- or C-terminal residues of the PRO211, PRO228, PRO538, PRO172 or PRO182. Derivatization with bifunctional agents is useful, for instance, for crosslinking PRO211, PRO228, PRO538, PRO172 or PRO182 to a water-insoluble support matrix or surface for use in the method for purifying anti-PRO211, anti-PRO228, anti-PRO538, anti-PRO172 or anti-PRO182 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl orthreonyl residues, methylation of the a-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO211, PRO228, PRO538, PRO172 or PRO182 polypeptide included within the scope of this invention comprises altering the native glycosylation pattern of the polypeptide. “Altering the native glycosylation pattern” is intended for purposes herein to mean deleting one or more carbohydrate moieties found in native sequence PRO211, PRO228, PRO538, PRO172 or PRO182 (either by removing the underlying glycosylation site or by deleting the glycosylation by chemical and/or enzymatic means), and/or adding one or more glycosylation sites that are not present in the native sequence PRO211, PRO228, PRO538, PRO172 or PRO182. In addition, the phrase includes qualitative changes in the glycosylation of the native proteins, involving a change in the nature and proportions of the various carbohydrate moieties present.

Addition of glycosylation sites to the PRO211, PRO228, PRO538, PRO172 or PRO182 polypeptide may be accomplished by altering the amino acid sequence. The alteration may be made, for example, by the addition of, or substitution by, one or more serine or threonine residues to the native sequence PRO211, PRO228, PRO538, PRO172 or PRO182 (for O-linked glycosylation sites). The PRO211, PRO228, PRO538, PRO172 or PRO182 amino acid sequence may optionally be altered through changes at the DNA level, particularly by mutating the DNA encoding the PRO211, PRO228, PRO538, PRO172 or PRO182 polypeptide at preselected bases such that codons are generated that will translate into the desired amino acids.

Another means of increasing the number of carbohydrate moieties on the PRO211, PRO228, PRO538, PRO172 or PRO182 polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the PRO211, PRO228, PRO538, PRO172 or PRO182 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO211, PRO228, PRO538, PRO172 or PRO182 comprises linking the PRO211, PRO228, PRO538, PRO172 or PRO182 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The PRO211, PRO228, PRO538, PRO172 or PRO182 polypeptide of the present invention may also be modified in a way to form a chimeric molecule comprising PRO211, PRO228, PRO538, PRO172 or PRO182 fused to another, heterologous polypeptide or amino acid sequence.

In one embodiment, such a chimeric molecule comprises a fusion of the PRO211, PRO228, PRO538, PRO172 or PRO182 polypeptide with a tag polypeptide which provides an epitope to which an anti-tag antibody can selectively bind. The epitope tag is generally placed at the amino- or carboxyl-terminus of the PRO211, PRO228, PRO538, PRO172 or PRO182 polypeptide. The presence of such epitope-tagged forms of the PRO211, PRO228, PRO538, PRO172 or PRO182 polypeptide can be detected using an antibody against the tag polypeptide. Also, provision of the epitope tag enables the PRO211, PRO228, PRO538, PRO172 or PRO182 polypeptide to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E 10 antibodies thereto [Evan et al., Molecular and Cellular Biology, 5:3610-3616(1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem, 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO211, PRO228, PRO538, PRO172 or PRO182 polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO211, PRO228, PRO538, PRO172 or PRO182 polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also, U.S. Pat. No. 5,428,130 issued Jun. 27, 1995.

D. Preparation of PRO211, PRO228, PRO538, PRO172 and PRO182

The description below relates primarily to production of PRO211, PRO228, PRO538, PRO172 or PRO182 by culturing cells transformed or transfected with a vector containing PRO211, PRO228, PRO538, PRO172 or PRO182 nucleic acid. It is, of course, contemplated that alternative methods, which are well known in the art, may be employed to prepare PRO211, PRO228, PRO538, PRO172 or PRO182. For instance, the PRO211, PRO228, PRO538, PRO172 or PRO182 polypeptide sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO211, PRO228, PRO538, PRO172 or PRO182 polypeptide may be chemically synthesized separately and combined using chemical or enzymatic methods to produce the full-length PRO211, PRO228, PRO538, PRO172 or PRO182 polypeptide.

1. Isolation of DNA Encoding PRO211, PRO228, PRO538, PRO172 or PRO182

DNA encoding PRO211, PRO228, PRO538, PRO172 or PRO182 may be obtained from a cDNA library prepared from tissue believed to possess the PRO211, PRO228, PRO538, PRO172 or PRO182 mRNA and to express it at a detectable level. Accordingly, human PRO211, PRO228, PRO538, PRO172 or PRO182 DNA can be conveniently obtained from a cDNA library prepared from human tissue, such as described in the Examples. The PRO211-, PRO228-, PRO538-, PRO172- or PRO182-encoding gene may also be obtained from a genomic library or by known synthetic procedures (e.g., automated nucleic acid synthesis).

Libraries can be screened with probes (such as antibodies to the PRO211, PRO228, PRO538, PRO172 or PRO182 or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO211, PRO228, PRO538, PRO172 or PRO182 is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO211, PRO228, PRO538, PRO172 or PRO182 production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see, Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO211-, PRO228-, PRO538-, PRO172- or PRO182-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published May 2, 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J.Bacteriol., 737 [1983]), K. fragilis (ATCC 12,424), K. bulgaricus (ATCC 16,045), K. wickeramii (ATCC 24,178), K waltii (ATCC 56,500), K. drosophilarum (ATCC 36,906; Van den Berg et al., Bio/Technology, 8:135 (1990)), K thermotolerans, and K. marxianus; yarrowia (EP 402,226); Pichia pastoris (EP 183,070; Sreekrishna et al., J. Basic Microbiol. 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs 269 (1982).

Suitable host cells for the expression of glycosylated PRO211, PRO228, PRO538, PRO172 or PRO182 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36:59(1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and use of a Replicable Vector

The nucleic acid (e.g., cDNA or genomic DNA) encoding PRO211, PRO228, PRO538, PRO172 or PRO182 may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO211, PRO228, PRO538, PRO172 or PRO182 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO211-, PRO228-, PRO538-, PRO172- or PRO182-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2 μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO211-, PRO228-, PRO538-, PRO172- or PRO182-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trpl gene present in the yeast plasmid YRp7 [Stinchcomb et al., Nature, 282:39 (1979); Kingsman et al., Gene, 7:141 (1979); Tschemper et al., Gene 10:157 (1980)]. The trpl gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO211-, PRO228-, PRO538-, PRO172- or PRO182-encoding nucleic acid sequence to direct mRNA synthesis. Promoters recognized by a variety of potential host cells are well known. Promoters suitable for use with prokaryotic hosts include the P-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO211, PRO228, PRO538, PRO172 or PRO182.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO211, PRO228, PRO538, PRO172 or PRO182 transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO211, PRO228, PRO538, PRO172 or PRO182 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO211, PRO228, PRO538, PRO172 or PRO182 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO211, PRO228, PRO538, PRO172 or PRO182.

Still other methods, vectors, and host cells suitable for adaptation to the synthesis of PRO211, PRO228, PRO538, PRO172 or PRO182 in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO211, PRO228, PRO538, PRO172 or PRO182 polypeptide or against a synthetic peptide based on the DNA sequences provided herein or against exogenous sequence fused to PRO211, PRO228, PRO538, PRO172 or PRO182 DNA and encoding a specific antibody epitope.

5. Purification of Polypeptide

Forms of PRO211, PRO228, PRO538, PRO172 or PRO182 may be recovered from culture medium or from host cell lysates. If membrane-bound, it can be released from the membrane using a suitable detergent solution (e.g., Triton-X 100) or by enzymatic cleavage. Cells employed in expression of PRO211, PRO228, PRO538, PRO172 or PRO182 can be disrupted by various physical or chemical means, such as freeze-thaw cycling, sonication, mechanical disruption, or cell lysing agents.

It may be desired to purify PRO211, PRO228, PRO538, PRO172 or PRO182 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO211, PRO228, PRO538, PRO172 or PRO182. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO211, PRO228, PRO538, PRO172 or PRO182 produced.

E. Antibodies

Some drug candidates for use in the compositions and methods of the present invention are antibodies and antibody fragments which mimic the biological activity of a PRO211, PRO228, PRO538, PRO172 or PRO182 polypeptide.

1. Polyclonal Antibodies

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO211, PRO228, PRO538, PRO172 or PRO182 polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bend to the immunizing agent. Alternatively, the lymphocytes may be immunized in vitro.

The immunizing agent will typically include the PRO211, PRO228, PRO538, PRO172 or PRO182 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp.59-103]. Immortalizedcell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myelomaand mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO211, PRO228, PRO538, PRO172 or PRO182. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al., and Boemer et al., are also available for the preparation of human monoclonal antibodies (Cole et al., Monoclonal Antibodies and Cancer Therapy, Alan R. Liss, p.77 (1985) and Boerner et al., J. Immunol., 147(1):86-95 (1991)]. Similarly, human antibodies can be made by the introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10: 779-783 (1992); Lonberg et al., Nature, 368: 856-859(1994); Morrison, Nature, 368: 812-13 (1994); Fishwild et al., Nature Biotechnology, 14:845-51 (1996); Neuberger, Nature Biotechnology, 14: 826 (1996); Lonberg and Huszar, Intern. Rev. Immunol., 13:65-93 (1995).

4. Bispecific Antibodies

Bispecific antibodies are monoclonal, preferably human or humanized, antibodies that have binding specificities for at least two different antigens. In the present case, one of the binding specificities is for the PRO211, PRO228, PRO538, PRO172 or PRO182, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol., 152:5368 (1994).

Antibodies with more than two valencies are contemplated. For example, trispecific antibodies can be prepared. Tutt et al., J. Immunol., 147:60 (1991).

Exemplary bispecific antibodies may bind to two different epitopes on a given PRO211, PRO228, PRO538, PRO172 or PRO182 polypeptide herein. Alternatively, an anti-PRO211, anti-PRO228, anti-PRO538, anti-PRO172 or anti-PRO182 polypeptide arm may be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD 16) so as to focus cellular defense mechanisms to the cell expressing the particular PRO211, PRO228, PRO538, PRO172 or PRO182 polypeptide. Bispecific antibodies may also be used to localize cytotoxic agents to cells which express a particular PRO211, PRO228, PRO538, PRO172 or PRO182 polypeptide. These antibodies possess a PRO211-, PRO228-, PRO538-, PRO172- or PRO182-binding arm and an arm which binds a cytotoxic agent or a radionuclide chelator, such as EOTUBE, DPTA, DOTA, or TETA. Another bispecific antibody of interest binds the PRO211, PRO228, PRO538, PRO172 or PRO182 polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the. Fc region, thereby allowing interchain disulfide bond formation in this region. The homodimeric antibody thus generated may have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al., Cancer Research, 53: 2560-2565 (1993). Alternatively, an antibody can be engineered that has dual Fc regions and may thereby have enhanced complement lysis and ADCC capabilities. See, Stevenson et al., Anti-Cancer Drug Design, 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents suchasN-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098(1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

F. Identification of Proteins Capable of Inhibiting Neoplastic Cell Growth or Proliferation

The proteins disclosed in the present application have been assayed in a panel of 60 tumor cell lines currently used in the investigational, disease-oriented, in vitro drug-discovery screen of the National Cancer Institute (NCI). The purpose of this screen is to identify molecules that have cytotoxic and/or cytostatic activity against different types of tumors. NCI screens more than 10,000 new molecules per year (Monks et al., J. Natl. Cancer Inst., 83:757-766 (1991); Boyd, Cancer: Princ. Pract. Oncol. Update, 3(10):1-12 ([1989]). The tumor cell lines employed in this study have been described in Monks et al., supra. The cell lines the growth of which has been significantly inhibited by the proteins of the present application are specified in the Examples.

The results have shown that the proteins tested show cytostatic and, in some instances and concentrations, cytotoxic activities in a variety of cancer cell lines, and therefore are useful candidates for tumor therapy.

Other cell-based assays and animal models for tumors (e.g., cancers) can also be used to verify the findings of the NCI cancer screen, and to further understand the relationship between the protein identified herein and the development and pathogenesis of neoplastic cell growth. For example, primary cultures derived from tumors in transgenic animals (as described below) can be used in the cell-based assays herein, although stable cell lines are preferred. Techniques to derive continuous cell lines from transgenic animals are well known in the art (see, e.g., Small et al., Mol. Cell. Biol., 5:642-648 [1985]).

G. Animal Models

A variety of well known animal models can be used to further understand the role of the molecules identified herein in the development and pathogenesis of tumors, and to test the efficacy of candidate therapeutic agents, including antibodies, and other agonists of the native polypeptides, including small molecule agonists. The in vivo nature of such models makes them particularly predictive of responses in human patients. Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. (See, e.g., PCT publication No. WO 97/33551, published Sep. 18, 1997).

Probably the most often used animal species in oncological studies are immunodeficient mice and, in particular, nude mice. The observation that the nude mouse with hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds., CRC Press, Inc., 1991.

The cells introduced into such animals can be derived from known tumor/cancer cell lines, such as, any of the above-listed tumor cell lines, and, for example, the B 104-1-1 cell line (stable NIH-3T3 cell line transfected with the neu protooncogene); ras-transfected NIH-3T3 cells; Caco-2 (ATCC HTB-37); a moderately well-differentiated grade II human colon adenocarcinoma cell line, HT-29 (ATCC HTB-38), or from tumors and cancers. Samples of tumor or cancer cells can be obtained from patients undergoing surgery, using standard conditions, involving freezing and storing in liquid nitrogen (Karmali et al., Br. J. Cancer, 48:689-696 [1983]).

Tumor cells can be introduced into animals, such as nude mice, by a variety of procedures. The subcutaneous (s.c.) space in mice is very suitable for tumor implantation. Tumors can be transplanted s.c. as solid blocks, as needle biopsies by use of a trochar, or as cell suspensions. For solid block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue. Boven and Winograd (1991), supra. Animal models of breast cancer can be generated, for example, by implanting rat neuroblastoma cells (from which the neu oncogen was initially isolated), or neu-transformed NIH-3T3 cells into nude mice, essentially as described by Drebin et al., Proc. Natl. Acad. Sci. USA, 83:9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research 54:4726-4728 (1994) and Too et al., Cancer Research, 55:681-684 (1995). This model is based on the so-called “METAMOUSE” sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21, and WEHI-164 are chemically induced fibrosarcomas of BALB/c female mice (DeLeo et al., J. Exp. Med., 146:720 [1977]), which provide a highly controllable model system for studying the anti-tumor activities of various agents (Palladino et al., J. Immunol. 138:4023-4032 [1987]). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small cell carcinoma of the lung (SCCL). This tumor can be introduced in normal mice upon injection of tumor fragments from an affected mouse or of cells maintained in culture (Zupi et al., Br. J. Cancer, 41, suppl. 4:309 [1980]), and evidence indicates that tumors can be started from injection of even a single cell and that a very high proportion of infected tumor cells survive. For further information about this tumor model see, Zacharski, Haemostasis, 16:300-320 [1986]).

One way of evaluating the efficacy of a test compound in an animal model on an implanted tumor is to measure the size of the tumor before and after treatment. Traditionally, the size of implanted tumors has been measured with a slide caliper in two or three dimensions. The measure limited to two dimensions does not accurately reflect the size of the tumor, therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals, Wu and Sheng eds., Basel, 1989, 301. It is noted, however, that necrosis and inflammatory responses following treatment may actually result in an increase in tumor size, at least initially. Therefore, these changes need to be carefully monitored, by a combination of a morphometric method and flow cytometric analysis.

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA, 82:6148-615 [1985]); gene targeting in embryonic stem cells (Thompson et al., Cell, 56:313-321 [1989]); electroporation of embryos (Lo, Mol. Cell. Biol., 3:1803-1814 [1983]); sperm-mediated gene transfer (Lavitrano et al., Cell, 57:717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89:6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

The efficacy of antibodies specifically binding the polypeptides identified herein and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis may merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination, biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chrondroma, leiomyosarcoma of dogs, cats, and baboons can also be tested. Of these mammary adenocarcinoma in dogs and cats is a preferred model as its appearance and behavior are very similar to those in humans. However, the use of this model is limited by the rare occurrence of this type of tumor in animals.

H. Screening Assays for Drug Candidates

Screening assays for drug candidates are designed to identify compounds that competitively bind or complex with the receptor(s) of the polypeptides identified herein, or otherwise signal through such receptor(s). Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art.

In binding assays, the interaction is binding and the complex formed can be isolated or detected in the reaction mixture. In a particular embodiment, a receptor of a polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular receptor, its interaction with that polypeptide can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London), 340:245-246 (1989); Chien et al., Proc. Natl. Acad. Sci. USA, 88:9578-9582 (1991)] as disclosed by Chevray and Nathans [Proc. Natl. Acad. Sci. USA, 89:5789-5793 (1991)]. Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

I. Pharmaceutical Compositions

The polypeptides of the present invention, agonist antibodies specifically binding proteins identified herein, as well as other molecules identified by the screening assays disclosed herein, can be administered for the treatment of tumors, including cancers, in the form of pharmaceutical compositions.

Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA, 90:7889-7893 [1993]).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

Therapeutic formulations of the polypeptides identified herein, or agonists thereof are prepared for storage by mixing the active ingredient having the desired degree of purity with optional pharmaceutically acceptable carriers, excipients or stabilizers (Remington's Pharmaceutical Sciences, 16th edition, Osol, A. ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organicacids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. ed. (1980).

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

J. Methods of Treatment

It is contemplated that the polypeptides of the present invention and their agonists, including antibodies, peptides, and small molecule agonists, may be used to treat various tumors, e.g., cancers. Exemplary conditions or disorders to be treated include benign or malignant tumors (e.g., renal, liver, kidney, bladder, breast, gastric, ovarian, colorectal, prostate, pancreatic, lung, vulval, thyroid, hepatic carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic and immunologic disorders. The anti-tumor agents of the present invention (including the polypeptides disclosed herein and agonists which mimic their activity, e.g., antibodies, peptides and small organic molecules), are administered to a mammal, preferably a human, in accord with known methods, such as intravenous administration as a bolus or by continuous infusion over a period of time, or by intramuscular, intraperitoneal, intracerobrospinal, intraocular, intraarterial, intralesional, subcutaneous, intraarticular, intrasynovial, intrathecal, oral, topical, or inhalation routes.

Other therapeutic regimens may be combined with the administration of the anti-cancer agents of the instant invention. For example, the patient to be treated with such anti-cancer agents may also receive radiation therapy. Alternatively, or in addition, a chemotherapeutic agent may be administered to the patient. Preparation and dosing schedules for such chemotherapeutic agents may be used according to manufacturers' instructions or as determined empirically by the skilled practitioner. Preparation and dosing schedules for such chemotherapy are also described in Chemotherapy Service, ed., M.C. Perry, Williams & Wilkins, Baltimore, Md. (1992). The chemotherapeutic agent may precede, or follow administration of the anti-tumor agent of the present invention, or may be given simultaneously therewith. The anti-cancer agents of the present invention may be combined with an anti-oestrogen compound such as tamoxifen or an anti-progesterone such as onapristone (see, EP 616812) in dosages known for such molecules.

It may be desirable to also administer antibodies against tumor associated antigens, such as antibodies which bind to the ErbB2, EGFR, ErbB3, ErbB4, or vascular endothelial factor (VEGF). Alternatively, or in addition, two or more antibodies binding the same or two or more different cancer-associated antigens may be co-administered to the patient. Sometimes, it may be beneficial to also administer one or more cytokines to the patient. In a preferred embodiment, the anti-cancer agents herein are co-administered with a growth inhibitory agent. For example, the growth inhibitory agent may be administered first, followed by the administration of an anti-cancer agent of the present invention. However, simultaneous administration or administration of the anti-cancer agent of the present invention first is also contemplated. Suitable dosages for the growth inhibitory agent are those presently used and may be lowered due to the combined action (synergy) of the growth inhibitory agent and the antibody herein.

For the prevention or treatment of disease, the appropriate dosage of an anti-tumor agent herein will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” in Toxicokinetics and New Drug Development, Yacobi et al., eds., Pergamon Press, New York 1989, pp. 42-96.

For example, depending on the type and severity of the disease, about 1 μg/kg to 15 mg/kg (e.g., 0.1-20 mg/kg) of an antitumor agent is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

K. Articles of Manufacture

In another embodiment of the invention, an article of manufacture containing materials useful for the diagnosis or treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is an anti-tumor agent of the present invention. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Isolation of cDNA clones Encoding PRO211 PRO228, PRO538, PRO172 and PRO182

(A) PRO211

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA28730. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA28730 consensus sequence oligonucleotides were synthesized: 1) to identify by PCR a cDNA library that contained the sequence of interest, and 2) for use as probes to isolate a clone of the full-length coding sequence for PRO211. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

forward PCR primer:

5′-AGAGTGTATCTCTGGCTACGC-3′ (SEQ ID NO:3)

reverse PCR primer:

5′-TAAGTCCGGCACATTACAGGTC-3′ (SEQ ID NO:4)

Additionally, a synthetic oligonucleotide hybridization probe was constructed from the consensus DNA28730 sequence which had the following nucleotide sequence: hybridization probe:

5′-AGGGAGCACGGACAGTGTGCAGATGTGGACGAGTGCTCACTAGCA-3′ (SEQ ID NO:5)

RNA for construction of the cDNA libraries was isolated from human fetal lung tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science 253:1278-1280 (1991)) in the unique XhoI and NotI sites.

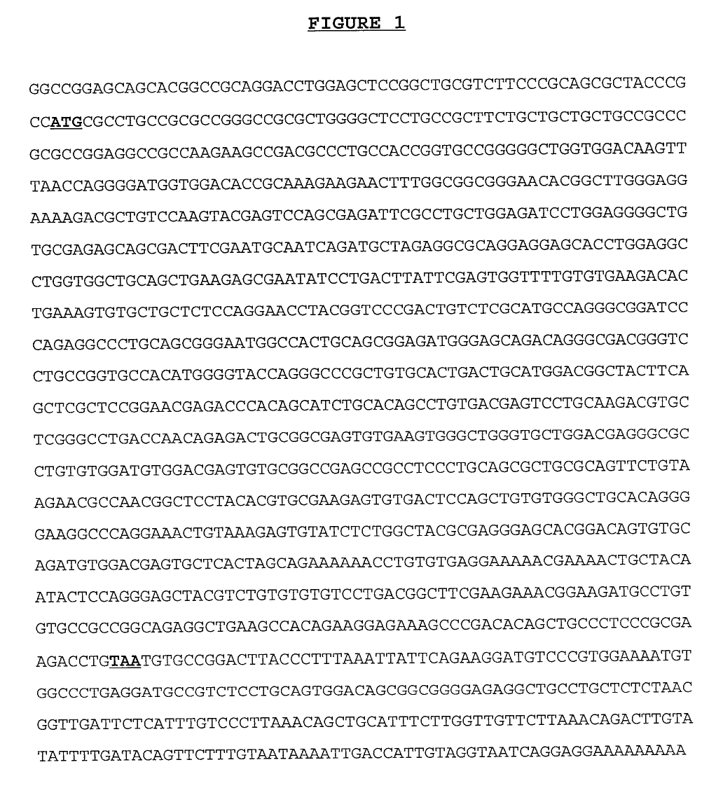

DNA sequencing of the clones isolated as described above gave the full-length DNA sequence for a full-length PRO211 polypeptide (designated herein as DNA32292-1131 [FIG. 1, SEQ ID NO:1]) and the derived protein sequence for that PRO211 polypeptide.

The full length clone identified above contained a single open reading frame with an apparent translational initiation site at nucleotide positions 65-67 and a stop signal at nucleotide positions 1124-1126 (FIG. 1, SEQ ID NO:1). The predicted polypeptide precursor is 353 amino acids long, has a calculated molecular weight of approximately 38,190 daltons. Analysis of the full-length PRO211 sequence shown in FIG. 2 (SEQ ID NO:2) evidences the presence of a variety of important polypeptide domains, wherein the locations given for those important polypeptide domains are approximate as described above. Analysis of the full-length PRO211 sequence evidenced the following: a signal peptide from about amino acid 1 to about amino acid 24; N-glycosylation sites from about amino acid 190 to about amino acid 194 and from about amino acid 251 to about amino acid 255; glycosaminoglycan attachment sites from about amino acid 149 to about amino acid 153 and from about amino acid 155 to about amino acid 159; a cAMP- and cGMP-dependent protein kinase phosphorylation site from about amino acid 26 to about amino acid 30; casein kinase II phosphorylation sites from about amino acid 58 to about amino acid 62, from about amino acid 66 to about amino acid 70, from about amino acid 86 to about amino acid 90, from about amino acid 197 to about amino acid 201, from about amino acid 210 to about amino acid 214, from about amino acid 255 to about amino acid 259, from about amino acid 295 to about amino acid 299, from about amino acid 339 to about amino acid 343, and from about amino acid 349 to about amino acid 353; a tyrosine kinase phosphorylation site from about amino acid 303 to about amino acid 310; N-myristoylation sites from about amino acid 44 to about amino acid 50, from about amino acid 54 to about amino acid 60, from about amino acid 55 to about amino acid 61, from about amino acid 81 to about amino acid 87, from about amino acid 150 to about amino acid 156, from about amino acid 158 to about amino acid 164, from about amino acid 164 to about amino acid 170, from about amino acid 252 to about amino acid 258, and from about amino acid 313 to about amino acid 319; an aspartic acid and asparagine hydroxylation site from about amino acid 308 to about amino acid 320; an EGF-like domain cysteine pattern signature from about amino acid 166 to about amino acid 178; and a leucine zipper pattern from about amino acid 94 to about amino acid 116.

Clone DNA32292-1131 has been deposited with ATCC on Sep. 16, 1997 and is assigned ATCC deposit no. 209258.

An analysis of the Dayhoff database (version 35.45 SwissProt 35), using the WU-BLAST2 sequence alignment analysis of the fill-length sequence shown in FIG. 2 (SEQ ID NO:2), evidenced sequence identity between the PRO211 amino acid sequence and human EGF.

(B) PRO228

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA28758. An EST proprietary to Genentech, Inc., designated herein as DNA21951, was employed in the consensus assembly. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA28758 consensus sequence oligonucleotides were synthesized: 1) to identify by PCR a cDNA library that contained the sequence of interest, and 2) for use as probes to isolate a clone of the full-length coding sequence for PRO228. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

forward PCR primer 1:

5′-GGTAATGAGCTCCATTACAG-3′ (SEQ ID NO:8)

forward PCR primer 2:

5′-GGAGTAGAAAGCGCATGG-3′ (SEQ ID NO:9)

forward PCR primer 3:

5′-CACCTGATACCATGAATGGCAG-3′ (SEQ ID NO:10)

reverse PCR primer 1:

5′-CGAGCTCGAATTAATTCG-3′ (SEQ ID NO:11)

reverse PCR primer 2:

5′-GGATCTCCTGAGCTCAGG-3′ (SEQ ID NO:12)

reverse PCR primer 3:

5′-CCTAGTTGAGTGATCCTTGTAAG-3′ (SEQ ID NO:13)

Additionally, a synthetic oligonucleotide hybridization probe was constructed from the consensus DNA28758 sequence which had the following nucleotide sequence: hybridization probe:

5′-ATGAGACCCACACCTCATGCCGCTGTAATCACCTGACACATTTTGCAATT-3′ (SEQ ID NO:14)

RNA for construction of the cDNA libraries was isolated from human fetal kidney tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

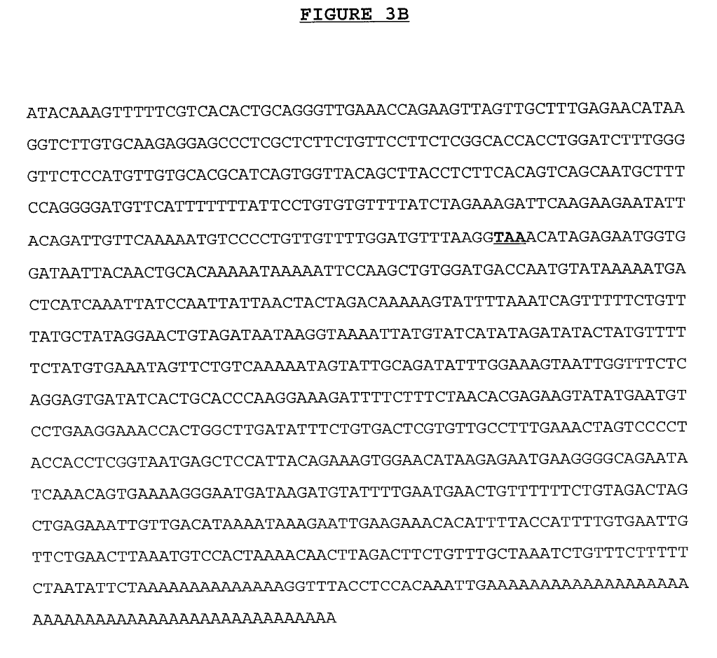

DNA sequencing of the clones isolated as described above gave the full-length DNA sequence for a full-length PRO228 polypeptide (designated herein as DNA33092-1202 [FIGS. 3A-B, SEQ ID NO:6]) and the derived protein sequence for that PRO228 polypeptide.

The full length clone identified above contained a single open reading frame with an apparent translational initiation site at nucleotide positions 24-26 and a stop signal at nucleotide positions 2094-2096 (FIGS. 3A-B, SEQ ID NO:6). The predicted polypeptide precursor is 690 amino acids long. Analysis of the full-length PRO228 sequence shown in FIG. 4 (SEQ ID NO:7) evidences the presence of a variety of important polypeptide domains, wherein the locations given for those important polypeptide domains are approximate as described above. Analysis of the full-length PRO228 sequence evidenced the following: a signal peptide from about amino acid 1 to about amino acid 19; transmembrane domains from about amino acid 430 to about amino acid 450, from about amino acid 465 to about amino acid 486, from about amino acid 499 to about amino acid 513, from about amino acid 535 to about amino acid 549, from about amino acid 573 to about amino acid 593, from about amino acid 619 to about amino acid 636, and from about amino acid 648 to about amino acid 664; N-glycosylation sites from about amino acid 15 to about amino acid 19, from about amino acid 21 to about amino acid 25, from about amino acid 64 to about amino acid 68, from about amino acid 74 to about amino acid 78, from about amino acid 127 to about amino acid 131, from about amino acid 177 to about amino acid 181, from about amino acid 188 to about amino acid 192, from about amino acid 249 to about amino acid 253, from about amino acid 381 to about amino acid 385, and from about amino acid 395 to about amino acid 399; a glycosaminoglycan attachment site from about amino acid 49 to about amino acid 53; a c-AMP- and cGMP-dependent protein kinase phosphorylation site from about amino acid 360 to about amino acid 364; casein kinase II phosphorylation sites from about amino acid 54 to about amino acid 58, from about amino acid 68 to about amino acid 72, from about amino acid 76 to about amino acid 80, from about amino acid 94 to about amino acid 98, from about amino acid 135 to about amino acid 139, from about amino acid 150 to about amino acid 154, from about amino acid 155 to about amino acid 159, from about amino acid 161 to about amino acid 165, from about amino acid 181 to about amino acid 185, from about amino acid 190 to about amino acid 194, from about amino acid 244 to about amino acid 248, from about amino acid 310 to about amino acid 314, from about amino acid 325 to about amino acid 329, from about amino acid 346 to about amino acid 350, and from about amino acid 608 to about amino acid 612; tyrosine kinase phosphorylation sites from about amino acid 36 to about amino acid 44 and from about amino acid 670 to about amino acid 677; N-myristoylation sites from about amino acid 38 to about amino acid 44, from about amino acid 50 to about amino acid 56, from about amino acid 52 to about amino acid 58, from about amino acid 80 to about amino acid 86, from about amino acid 382 to about amino acid 388, from about amino acid 388 to about amino acid 394, from about amino acid 434 to about amino acid 440, from about amino acid 480 to about amino acid 486, and from about amino acid 521 to about amino acid 527; and an aspartic acid and asparagine hydroxylation site from about amino acid 75 to about amino acid 87.

Clone DNA33092-1202 has been deposited with ATCC on Oct. 28, 1997 and is assigned ATCC deposit no. 209420.

An analysis of the Dayhoff database (version 35.45 SwissProt 35), using the WU-BLAST2 sequence alignment analysis of the full-length sequence shown in FIG. 4 (SEQ ID NO:7), evidenced significant sequence identity between the PRO228 amino acid sequence and the secretin related proteins CD97 and EMR1 as well as the secretin member, latrophilin, thereby indicating that PRO228 may be a new member of the secretin related proteins.

(C) PRO538

An expressed sequence tag (EST) DNA database and a proprietary EST database (LIFESEQ®, Incyte Pharmaceuticals, Palo Alto, Calif.) was searched and an Incyte EST (INC3574209) was identified which had 61% sequence identity to murine GFRα3.

RNA for construction of cDNA libraries was then isolated from human fetal lung tissue. The cDNA libraries used to isolate the cDNA clones encoding human PRO538 were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI.

Oligonucleotides probes based upon the above described EST sequence were then synthesized: 1) to identify by PCR a cDNA library that contained the sequence of interest, and 2) for use as probes to isolate a clone of the full-length coding sequence for PRO538. Forward and reverse PCR primers generally range from 20 to 30 nucleotides and are often designed to give a PCR product of about 100-1000 bp in length. The probe sequences are typically 40-55 bp in length. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

The oligonucleotide probes employed were as follows:

forward PCR primer:

5′-GCCTCTCGCAGCCGGAGACC-3′ (SEQ ID NO:17)

reverse PCR primer:

5′-CAGGTGGGATCAGCCTGGCAC-3′ (SEQ ID NO:18)

hybridization probe:

5′-TCTCGCAGCCGGAGACCCCCTTCCCACAGAAAGCCGACTCA-3′ (SEQ ID NO:19)

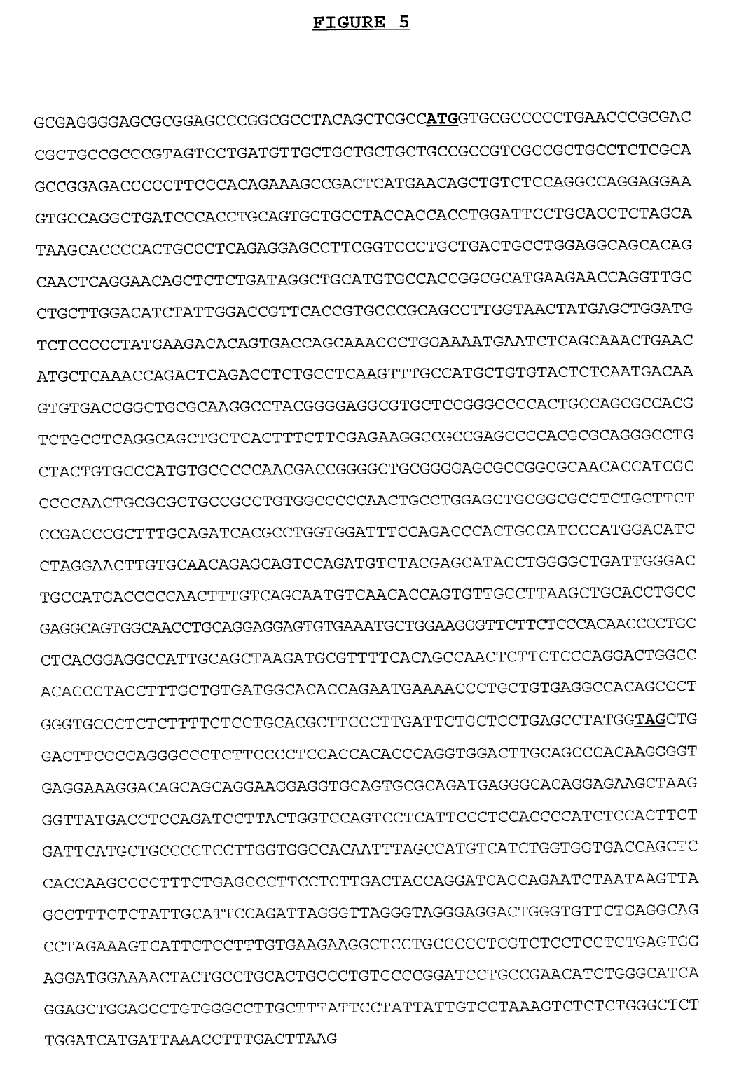

Pure positive clones were obtained after colony purification and secondary screening. Five positive clones were identified. Two of the isolated clones were sequenced. These cDNA sequences were designated DNA48613-1268 and DNA48614-1268. A full length clone for DNA48613-1268 was identified that contained a single open reading frame with an apparent translational initiation site at nucleotide positions 38-40 and a stop signal at nucleotide positions 1238-1240 (FIG. 5, SEQ ID NO:15). The predicted polypeptide precursor is 400 amino acids long, has a calculated molecular weight of approximately 44,511 daltons and an estimated pi of approximately 8.15. A comparison of the amino acid sequence of DNA48614-1268 to the amino acid sequence of DNA48613-1268 (FIG. 5; SEQ ID NO:15), revealed it to be an alternatively spliced form of DNA48613-1268, with a 30 amino acid deletion (amino acids 127-157, counting from the initiation methionine).

Analysis of the full-length PRO538 sequence shown in FIG. 6 (SEQ ID NO:16) evidences the presence of a variety of important polypeptide domains, wherein the locations given for those important polypeptide domains are approximate as described above. Analysis of the full-length PRO538 sequence evidenced the following: a signal peptide from about amino acid 1 to about amino acid 26; a transmembrane domain from about amino acid 379 to about amino acid 395; N-glycosylation sites from about amino acid 95 to about amino acid 99, from about amino acid 148 to about amino acid 152, and from about amino acid 309 to about amino acid 313; a cAMP- and cGMP-dependent protein kinase phosphorylation site from about amino acid 231 to about amino acid 235; casein kinase II phosphorylation sites from about amino acid 134 to about amino acid 138, from about amino acid 170 to about amino acid 174, and from about amino acid 202 to about amino acid 206; N-myristoylation sites from about amino acid 279 to about amino acid 285 and from about amino acid 294 to about amino acid 300; and prokaryotic membrane lipoprotein lipid attachment sites from about amino acid 306 to about amino acid 317 and from about amino acid 379 to about amino acid 390.

Clone DNA48613-1268 has been deposited with ATCC on Apr. 7, 1998 and is assigned ATCC deposit no. 209752.

As discussed below, a sequence comparison of the full-length sequence shown in FIG. 6 (SEQ ID NO:16) encoded by DNA48613-1268 to the sequences of human GFRα1 and GFRα2 indicated that the human protein is a new member of the GFRα receptor family, and is a human homolog of murine GFRα3. Accordingly, DNA48613-1268 encodes a protein designated as human GFRα3, and DNA48614-1268 encodes its splice variant.

An analysis of the Dayhoff database (version 35.45 SwissProt 35), using the BLAST-2 and FastA sequence alignment analysis of the full-length sequence of PRO538 shown in FIG. 6 (SEQ ID NO:16) and other GFRA family members is provided in Table 4.

TABLE 4
Sequence Identity Between Members of the GFRα Family

| Proteins Compared | Percent Identity |
|---|---|
| rGFRα1 versus hGFRα1 | 92% |
| rGFRα2 versus hGFRα2 | 94% |
| mGFRα3 versus | 77% |
| hGFRα3 | |
| hGFRα3 versus hGFRα1 | 34% |
| hGFRα3 versus hGFRα2 | 34% |
| hGFRα1 versus hGFRα2 | 48% |

From the sequence comparisons it can be seen that human GFRα3 (PRO538) is less related to its rodent homolog than is either GFRα1 or GFRα2. In addition, GFRα3 (PRO538) appears to be more distantly related to GFRα1 and GFRα2 than GFRα1 and GFRα2 are to each other.

(D) PRO172

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA28765. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA28765 consensus sequence oligonucleotides were synthesized: 1) to identify by PCR a cDNA library that contained the sequence of interest, and 2) for use as probes to isolate a clone of the full-length coding sequence for PRO172. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

forward PCR primer:

5′-GGATCTCGAGAACAGCTACTCC-3′ (SEQ ID NO:22)

reverse PCR primer:

5′-TCGTCCACGTTGTCGTCACATG-3′ (SEQ ID NO:23)

Additionally, a synthetic oligonucleotide hybridization probe was constructed from the consensus DNA28765 sequence which had the following nucleotide sequence: hybridization probe:

5′-AAATCTGTGAATTGAGTGCCATGGACCTGTTGCGGACGGCCCTTGCTT-3′ (SEQ ID NO:24)

RNA for construction of the cDNA libraries was isolated from human fetal kidney tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

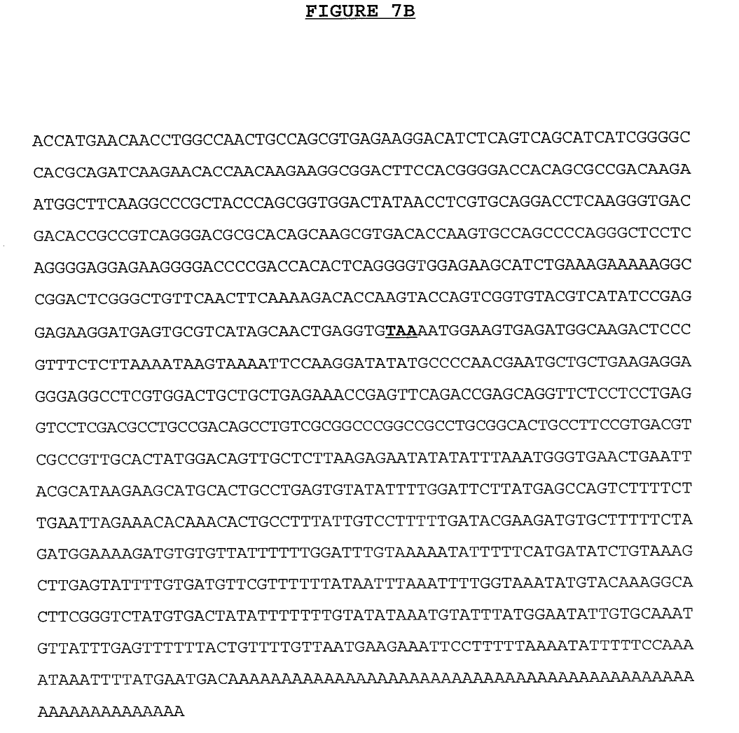

DNA sequencing of the clones isolated as described above gave the full-length DNA sequence for a full-length PRO172 polypeptide (designated herein as DNA35916-1161 [FIGS. 7A-B, SEQ ID NO:20]) and the derived protein sequence for that PRO172 polypeptide.

The full length clone identified above contained a single open reading frame with an apparent translational initiation site at nucleotide positions 38-40 and a stop signal at nucleotide positions 2207-2209 (FIGS. 7A-B, SEQ ID NO:20). The predicted polypeptide precursor is 723 amino acids long. Analysis of the full-length PRO172 sequence shown in FIG. 8 (SEQ ID NO:21) evidences the presence of a variety of important polypeptide domains, wherein the locations given for those important polypeptide domains are approximate as described above. Analysis of the full-length PRO172 sequence evidenced the following: a signal peptide from about amino acid 1 to about amino acid 21; a transmembrane domain from about amino acid 548 to about amino acid 568; an N-glycosylation site from about amino acid 477 to about amino acid 481; a cAMP- and cGMP-dependent protein kinase phosphorylation site from about amino acid 660 to about amino acid 664; casein kinase II phosphorylation sites from about amino acid 93 to about amino acid 97, from about amino acid 131 to about amino acid 135, from about amino acid 154 to about amino acid 158, from about amino acid 203 to about amino acid 207, from about amino acid 342 to about amino acid 346, from about amino acid 344 to about amino acid 348, from about amino acid 369 to about amino acid 373, from about amino acid 457 to about amino acid 461, from about amino acid 483 to about amino acid 487, from about amino acid 495 to about amino acid 499, from about amino acid 659 to about amino acid 663, from about amino acid 670 to about amino acid 674, from about amino acid 671 to about amino acid 675, and from about amino acid 698 to about amino acid 702; tyrosine kinase phosphorylation sites from about amino acid 176 to about amino acid 185 and from about amino acid 252 to about amino acid 261; N-myristoylation sites from about amino acid 2 to about amino acid 8, from about amino acid 37 to about amino acid 43, from about amino acid 40 to about amino acid 46, from about amino acid 98 to about amino acid 104, from about amino acid 99 to about amino acid 105, from about amino acid 262 to about amino acid 268, from about amino acid 281 to about amino acid 287, from about amino acid 282 to about amino acid 288, from about amino acid 301 to about amino acid 307, from about amino acid 310 to about amino acid 316, from about amino acid 328 to about amino acid 334, from about amino acid 340 to about amino acid 346, from about amino acid 378 to about amino acid 384, from about amino acid 3187 to about amino acid 393, from about amino acid 512 to about amino acid 518, from about amino acid 676 to about amino acid 682, from about amino acid 683 to about amino acid 689, and from about amino acid 695 to about amino acid 701; aspartic acid and asparagine hydroxylation sites from about amino acid 343 to about amino acid 355, from about amino acid 420 to about amino acid 432, and from about amino acid 458 to about amino acid 480; a prokaryotic membrane lipoprotein lipid attachment site from about amino acid 552 to about amino acid 563; and EGF-like domain cysteine pattern signatures from about amino acid 243 to about amino acid 255, from about amino acid 274 to about amino acid 286, from about amino acid 314 to about amino acid 326, from about amino acid 352 to about amino acid 364, from about amino acid 391 to about amino acid 403, from about amino acid 429 to about amino acid 441, from about amino acid 467 to about amino acid 479, and from about amino acid 505 to about amino acid 517.

Clone DNA35916-1161 has been deposited with ATCC on Oct. 28, 1997 and is assigned ATCC deposit no. 209419.

An analysis of the Dayhoff database (version 35.45 SwissProt 35), using the BLAST and FastAsequence alignment analysis of the full-length sequence shown in FIG. 8 (SEQ ID NO:21), evidenced 89% sequence identity between the PRO172 amino acid sequence and delta-1 mouse protein.

(E) PRO182

An expressed sequence tag (EST) DNA database and a proprietary EST database (LIFESEQ®, Incyte Pharmaceuticals, Palo Alto, Calif.) was searched and two EST sequences were identified (Incyte EST INC2328985 and Incyte EST INC778319), each having approximately 40% homology to a region of the relaxin nucleic acid sequence, and representing sequences within a gene of an insulin-like polypeptide. The EST corresponding to INC778319 was used to clone the full-length PRO182 gene.

RNA for construction of cDNA libraries was then isolated from human uterine tissue. The cDNA libraries used to isolate the cDNA clones encoding human PRO182 were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI.

Oligonucleotides probes based upon the above described EST sequence were then synthesized: 1) to identify by PCR a cDNA library that contained the sequence of interest, and 2) for use as probes to isolate a clone of the full-length coding sequence for PRO182. Forward and reverse PCR primers generally range from 20 to 30 nucleotides and are often designed to give a PCR product of about 100-1000 bp in length. The probe sequences are typically 40-55 bp in length. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

The oligonucleotide probes employed were as follows:

5′-CACATTCAGTCCTCAGCAAAATGAA-3′ (SEQ ID NO:27)

5′-GAGAATAAAAACAGAGTGAAAATGGAGCCCTTCATTTTGC-3′ (SEQ ID NO:28)

5′-CTCAGCTTGCTGAGCTTGAGGGA-3′ (SEQ ID NO:29)

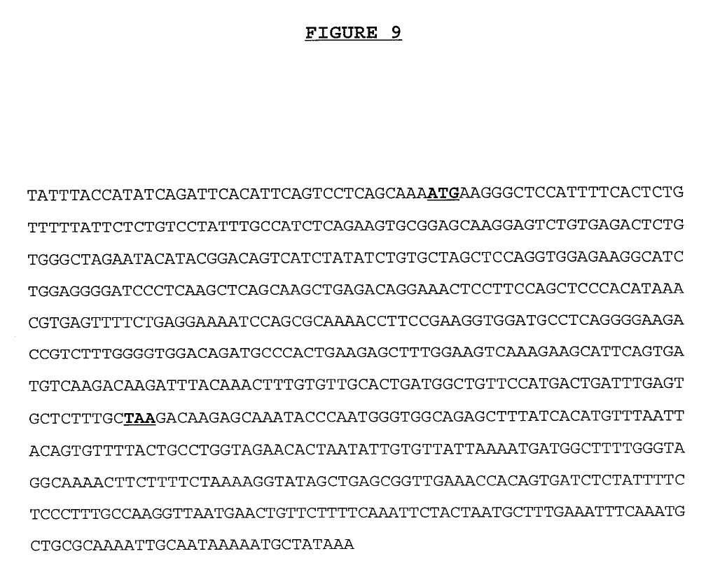

A full length clone for DNA27865-1091 was identified that contained a single open reading frame with an apparent translational initiation site at nucleotide positions 39-41 and a stop signal at nucleotide positions 444-446 (FIG. 9, SEQ ID NO:25). The predicted polypeptide precursor is 135 amino acids long.

Analysis of the full-length PRO182 sequence shown in FIG. 10 (SEQ ID NO:26) evidences the presence of a variety of important polypeptide domains, wherein the locations given for those important polypeptide domains are approximate as described above. Analysis of the full-length PRO182 sequence evidenced the following: a signal peptide from about amino acid 1 to about amino acid 18; a cAMP- and cGMP-dependent protein kinase phosphorylation site from about amino acid 107 to about amino acid 111; casein kinase II phosphorylation sites from about amino acid 88 to about amino acid 92, from about amino acid 113 to about amino acid 117, and from about amino acid 127 to about amino acid 131; N-myristoylation sites from about amino acid 3 to about amino acid 9, from about amino acid 52 to about amino acid 58, from about amino acid 96 to about amino acid 102, and from about amino acid 125 to about amino acid 131; and an insulin family signature from about amino acid 121 to about amino acid 136.

Clone DNA27865-1091 has been deposited with ATCC on Sep. 23, 1997 and is assigned ATCC deposit no. 209296.

An analysis of the Dayhoff database (version 35.45 SwissProt 35), using the WU-BLAST2 sequence alignment analysis of the full-length sequence shown in FIG. 10 (SEQ ID NO:26) evidenced sequence identity between the PRO182 amino acid sequence and a human insulin-like polypeptide, thus indicating that PRO182 is a novel human insulin-like protein.

### Example 2

### Expression of PRO211, PRO228, PRO538, PRO172 or PRO182 in E. coli

This example illustrates preparation of an unglycosylated form of PRO211, PRO228, PRO538, PRO172 or PRO182 by recombinant expression in E. coli.

The DNA sequence encoding PRO211, PRO228, PRO538, PRO172 or PRO182 is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a poly-His leader (including the first six STII codons, poly-His sequence, and enterokinase cleavage site), the PRO211, PRO228, PRO538, PRO172 or PRO182 coding region, lambda transcriptional terminator, and an argu gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO211, PRO228, PRO538, PRO172 or PRO182 protein can then be purified using a metal chelating column under conditions that allow tight binding of the protein.

PRO211, PRO228, PRO538, PRO172or PRO182 maybe expressed in E. coli in a poly-His tagged form, using the following procedure. The DNA encoding PRO211, PRO228, PRO538, PRO172 or PRO182 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(lacIq). Transformants are first grown in LB containing 50 mg/ml carbenicillin at 30° C. with shaking until an OD₆₀₀ of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 ml water, as well as 110 mM MPOS, pH 7.3,0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni²⁺-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros RI/H reversed phase column using a mobile buffer of 0.1% TFA with elution with a gradient of acetonitrile from 10 to 80%. Aliquots of fractions with A₂₈₀ absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO211, PRO228, PRO538, PRO172 or PRO182 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 3

### Expression of PRO211, PRO228, PRO538, PRO172 or PRO182 in mammalian cells

This example illustrates preparation of a potentially glycosylated form of PRO211, PRO228, PRO538, PRO172 or PRO182 by recombinant expression in mammalian cells.

The vector, pRK5 (see EP 307,247, published Mar. 15, 1989), is employed as the expression vector. Optionally, the PRO211, PRO228, PRO538, PRO172 or PRO182 DNA is ligated into pRK5 with selected restriction enzymes to allow insertion of the PRO211, PRO228, PRO538, PRO172 or PRO182 DNA using ligation methods such as described in Sambrook et al., supra. The resulting vector is called pRK5-PRO211, pRK5-PRO228, pRK5-PRO538, pRK5-PRO172 or pRK5-PRO182.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO211, pRK5-PRO228, pRK5-PRO538, pRK5-PRO172 or pRK5-PRO182 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 ,1 of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of the PRO211, PRO228, PRO538, PRO172 or PRO182 polypeptide. The cultures containing transfected cells may undergo further incubation (in serum free medium) and the medium is tested in selected bioassays.

In an alternative technique, PRO211, PRO228, PRO538, PRO172 or PRO182 may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO211, pRK5-PRQ228, pRK5-PRO538, pRK5-PRO172 or pRK5-PRO182 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO211, PRO228, PRO538, PRO172 or PRO182 can then be concentrated and purified by any selected method, such as dialysis and/or column chromatography.

In another embodiment, PRO211, PRO228, PRO538, PRO172 or PRO182 can be expressed in CHO cells. The pRK5-PRO211, pRK5-PRO228, pRK5-PRO538, pRK5-PRO172 or pRK-5-PRO182 can be transfected into CHO cells using known reagents such as CaPO₄ or DEAE-dextran. As described above, the cell cultures can be incubated, and the medium replaced with culture medium (alone) or medium containing a radiolabel such as 35S-methionine. After determining the presence of a PRO211, PRO228, PRO538, PRO172 or PRO182 polypeptide, the culture medium may be replaced with serum free medium. Preferably, the cultures are incubated for about 6 days, and then the conditioned medium is harvested. The medium containing the expressed PRO211, PRO228, PRO538, PRO172 or PRO182 polypeptide can then be concentrated and purified by any selected method.

Epitope-tagged PRO211, PRO228, PRO538, PRO172 or PRO182 may also be expressed in host CHO cells. The PRO211, PRO228, PRO538, PRO172 or PRO182 may be subcloned out of the pRK5 vector. The subclone insert can undergo PCR to fuse in frame with a selected epitope tag such as a poly-His tag into a Baculovirus expression vector. The poly-His tagged PRO211, PRO228, PRO538, PRO172 or PRO182 insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged PRO211, PRO228, PRO538, PRO172 or PRO182 can then be concentrated and purified by any selected method, such as by Ni²⁺-chelate affinity chromatography.

PRO211, PRO228, PRO538, PRO172 or PRO182 may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g., extracellular domains) of the respective proteins are fused to an IgG1 constant region sequence containing the hinge, CH2 and CH2 domains and/or as a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used in expression in CHO cells is as described in Lucas et al., Nucl. Acids Res., 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into a water bath and mixed by vortexing. The contents are pipetted into a centrifuge tube containing 10 mls of media and centrifuged at 1000 rpm for 5 minutes. The supernatant is aspirated and the cells are resuspended in 10 ml of selective media (0.2 μm filtered PS20 with 5% 0.2 μm diafiltered fetal bovine serum). The cells are then aliquoted into a 100 ml spinner containing 90 ml of selective media. After 1-2 days, the cells are transferred into a 250 ml spinner filled with 150 ml selective growth medium and incubated at 37° C. After another 2-3 days, 250 ml, 500 ml and 2000 ml spinners are seeded with 3×10⁵ cells/ml. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/ml. On day 0, the cell number and pH is determined. On day 1, the spinner is sampled and sparging with filtered air is commenced. On day 2, the spinner is sampled, the temperature shifted to 33° C., and 30 ml of 500 g/L glucose and 0.6 ml of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability drops below 70%, the cell culture is harvested by centrifugation and filtering through a 0.22 μm filter. The filtrate is either stored at 4° C. or immediately loaded onto columns for purification.

For the poly-His tagged constructs, the proteins are purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which has been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

PRO211, PRO172 and PRO182 were stably expressed in CHO cells by the above described method. In addition, PRO172 was expressed in CHO cells by the transient expression procedure.

### Example 4

Expression of PRO211, PRO228, PRO538, PRO172 or PRO182 in Yeast

The following method describes recombinant expression of PRO211, PRO228, PRO538, PRO172 or PRO182 in yeast.

First, yeast expression vectors are constructed for intracellular production or secretion of PRO211, PRO228, PRO538, PRO172 or PRO182 from the ADH2/GAPDH promoter. DNA encoding PRO211, PRO228, PRO538, PRO172 or PRO182 and the promoter is inserted into suitable restriction enzyme sites in the selected plasmid to direct intracellular expression of PRO211, PRO228, PRO538, PRO172 or PRO182. For secretion, DNA encoding PRO211, PRO228, PRO538, PRO172 or PRO182 can be cloned into the selected plasmid, together with DNA encoding the ADH2/GAPDH promoter, a native PRO211, PRO228, PRO538, PRO172 or PRO182 signal peptide or other mammalian signal peptide, or, for example, a yeast alpha-factor or invertase secretory signal/leader sequence, and linker sequences (if needed) for expression of PRO211, PRO228, PRO538, PRO172 or PRO182.

Yeast cells, such as yeast strain AB 110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO211, PRO228, PRO538, PRO172 or PRO182 can subsequently be isolated and purified by removing the yeast cells from the fermentation medium by centrifugation and then concentrating the medium using selected cartridge filters. The concentrate containing PRO211, PRO228, PRO538, PRO172 or PRO182 may further be purified using selected column chromatography resins.

### Example 5

Expression of PRO211, PRO228, PRO538, PRO172 or PRO182 in Baculovirus-Infected Insect Cells

The following method describes recombinant expression in Baculovirus-infected insect cells.

The sequence coding for PRO211, PRO228, PRO538, PRO172 or PRO182 is fused upstream of an epitope tag contained within a baculovirus expression vector. Such epitope tags include poly-His tags and immunoglobulin tags (like Fc regions of IgG). A variety of plasmids may be employed, including plasmids derived from commercially available plasmids such as pVL 1393 (Novagen). Briefly, the sequence encoding PRO211, PRO228, PRO538, PRO172 or PRO182 or the desired portion of the coding sequence of PRO211, PRO228, PRO538, PRO172 or PRO182 (such as the sequence encoding the extracellular domain of a transmembrane protein or the sequence encoding the mature protein if the protein is extracellular) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“S f9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-His tagged PRO211, PRO228, PRO538, PRO172 or PRO182 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 ml Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 mm filter. A Ni²⁺-NTA agarose column (commercially available from Qiagen) is prepared with a bed volume of 5 ml, washed with 25 ml of water and equilibrated with 25 ml of loading buffer. The filtered cell extract is loaded onto the column at 0.5 ml per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM imidazole gradient in the secondary wash buffer. One ml fractions are collected and analyzed by SDS-PAGE and silver staining or Western blot with Ni²⁺-NTA-conjugated to alkaline phosphatase (Qiagen). Fractions containing the eluted His₁₀-tagged PRO211, PRO228, PRO538, PRO172 or PRO182, respectively, are pooled and dialyzed against loading buffer.

Alternatively, purification of the IgG tagged (or Fc tagged) PRO211, PRO228, PRO538, PRO172 or PRO182 can be performed using known chromatography techniques, including for instance, Protein A or protein G column chromatography.

Following PCR amplification, the respective coding sequences are subcloned into a baculovirus expression vector (pb.PH.IgG for IgG fusions and pb.PH.His.c for poly-His tagged proteins), and the vector and Baculogold® baculovirus DNA (Pharmingen) are co-transfected into 105 Spodoptera frugiperda (“Sf9”) cells (ATCC CRL 1711), using Lipofectin (Gibco BRL). pb.PH.IgG and pb.PH.His are modifications of the commercially available baculovirus expression vector pVL1393 (Pharmingen), with modified polylinker regions to include the His or Fc tag sequences. The cells are grown in Hink's TNM-FH medium supplemented with 10% FBS (Hyclone). Cells are incubated for 5 days at 28° C. The supernatant is harvested and subsequently used for the first viral amplification by infecting Sf9 cells in Hink's TNM-FH medium supplemented with 10% FBS at an approximate multiplicity of infection (MOI) of 10. Cells are incubated for 3 days at 28° C. The supernatant is harvested and the expression of the constructs in the baculovirus expression vector is determined by batch binding of 1 ml of supernatant to 25 ml of Ni²-NTA beads (QIAGEN) for histidine tagged proteins or Protein-A Sepharose CL-4B beads (Pharmacia) for IgG tagged proteins followed by SDS-PAGE analysis comparing to a known concentration of protein standard by Coomassie blue staining.

The first viral amplification supernatant is used to infect a spinner culture (500 ml) of Sf9 cells grown in ESF-921 medium (Expression Systems LLC) at an approximate MOI of 0.1. Cells are incubated for 3 days at 28° C. The supernatant is harvested and filtered. Batch binding and SDS-PAGE analysis is repeated, as necessary, until expression of the spinner culture is confirmed.

The conditioned medium from the transfected cells (0.5 to 3 L) is harvested by centrifugation to remove the cells and filtered through 0.22 micron filters. For the poly-His tagged constructs, the protein construct is purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at-80° C.

Immunoadhesin (Fc containing) constructs of proteins are purified from the conditioned media as follows. The conditioned media is pumped onto a 5 ml Protein A column (Pharmacia) which has been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 ml of 1 M Tris buffer, pH 9. The highly purified protein is subsequently desalted into storage buffer as described above for the poly-His tagged proteins. The homogeneity of the proteins is verified by SDS polyacrylamide gel (PEG) electrophoresis and N-terminal amino acid sequencing by Edman degradation.

PRO228, PRO538 and PRO172 were expressed in baculovirus infected Sf9 insect cells.

Alternatively, a modified baculovirus procedure may be used incorporating high-5 cells. In this procedure, the DNA encoding the desired sequence is amplified with suitable systems, such as Pfu (Stratagene), or fused upstream (5′-of) of an epitope tag contained with a baculovirus expression vector. Such epitope tags include poly-His tags and immunoglobulin tags (like Fc regions of IgG). A variety of plasmids may be employed, including plasmids derived from commercially available plasmids such as pIE1-1 (Novagen). The pIE1-1 and pIE1-2 vectors are designed for constitutive expression of recombinant proteins from the baculovirus ie1 promoter in stably-transformed insect cells (1). The plasmids differ only in the orientation of the multiple cloning sites and contain all promoter sequences known to be important for ie1-mediated gene expression in uninfected insect cells as well as the hr5 enhancer element. pIE1-1 and pIE 1-2 include the translation initiation site and can be used to produce fusion proteins. Briefly, the desired sequence or the desired portion of the sequence (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subcloned into the expression vector. For example, derivatives of pIE1-1 can include the Fc region of human IgG (pb.PH.IgG) or an 8 histidine (pb.PH.His) tag downstream (3′-of) the desired sequence. Preferably, the vector construct is sequenced for confirmation.

High-5 cells are grown to a confluency of 50% under the conditions of, 27° C., no CO₂, NO pen/strep. For each 150 mm plate, 30 μg of pIE based vector containing these quence is mixed with 1 ml Ex-Cell medium (Media: Ex-Cell 401+1/100 L-Glu JRH Biosciences #14401-78P (note: this media is light sensitive)), and in a separate tube, 100 μl of Cell Fectin (CellFECTIN (Gibco BRL #10362-010) (vortexed to mix)) is mixed with 1 ml of Ex-Cell medium. The two solutions are combined and allowed to incubate at room temperature for 15 minutes. 8 ml of Ex-Cell media is added to the 2 ml of DNA/CellFECTIN mix and this is layered on high-5 cells that have been washed once with Ex-Cell media. The plate is then incubated in darkness for 1 hour at room temperature. The DNA/CellFECTIN mix is then aspirated, and the cells are washed once with Ex-Cell to remove excess CellFECTIN, 30 ml of fresh Ex-Cell media is added and the cells are incubated for 3 days at 28° C. The supernatant is harvested and the expression of the sequence in the baculovirus expression vector is determined by batch binding of 1 ml of supernatent to 25 ml of Ni²⁺-NTA beads (QIAGEN) for histidine tagged proteins or Protein-A Sepharose CL-4B beads (Pharmacia) for IgG tagged proteins followed by SDS-PAGE analysis comparing to a known concentration of protein standard by Coomassie blue staining.

The conditioned media from the transfected cells (0.5 to 3 L) is harvested by centrifugation to remove the cells and filtered through 0.22 micron filters. For the poly-His tagged constructs, the protein comprising the sequence is purified using a Ni²⁺-NTA column (Qiagen). Before purification, imidazole is added to the conditioned media to a concentration of 5 mM. The conditioned media is pumped onto a 6 ml Ni²⁺-NTA column equilibrated in 20 mM Hepes, pH 7.4, buffer containing 0.3 M NaCl and 5 mM imidazole at a flow rate of 4-5 ml/min. at 48° C. After loading, the column is washed with additional equilibration buffer and the protein eluted with equilibration buffer containing 0.25 M imidazole. The highly purified protein is then subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc containing) constructs of proteins are purified from the conditioned media as follows. The conditioned media is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 ml of 1 M Tris buffer, pH 9. The highly purified protein is subsequently desalted into storage buffer as described above for the poly-His tagged proteins. The homogeneity of the sequence is assessed by SDS polyacrylamide gels and by N-terminal amino acid sequencing by Edman degradation and other analytical procedures as desired or necessary.

PRO211, PRO228, PRO538, PRO172 and PRO182 were expressed using the above baculovirus procedure employing high-5 cells.

### Example 6

### Preparation of Antibodies that Bind PRO211 PRO228, PRO538, PRO172 or PRO182

This example illustrates preparation of monoclonal antibodies which can specifically bind PRO211, PRO228, PRO538, PRO172 or PRO182.

Techniques for producing the monoclonal antibodies are known in the art and are described, for instance, in Goding, supra. Immunogens that maybe employed include purified PRO211, PRO228, PRO538, PRO172 or PRO182, fusion proteins containing PRO211, PRO228, PRO538, PRO172 or PRO182, and cells expressing recombinant PRO211, PRO228, PRO538, PRO172 or PRO182 on the cell surface. Selection of the immunogen can be made by the skilled artisan without undue experimentation.

Mice, such as Balb/c, are immunized with the PRO211, PRO228, PRO538, PRO172 or PRO182 immunogen emulsified incomplete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, MT) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO211, anti-PRO228, anti-PRO538, anti-PRO172 or anti-PRO182 antibodies.

After a suitable antibody titer has been detected, the animals “positive” for antibodies can be injected with a final intravenous injection of PRO211, PRO228, PRO538, PRO172 or PRO182. Three to four days later, the mice are sacrificed and the spleen cells are harvested. The spleen cells are then fused (using 35% polyethylene glycol) to a selected murine myeloma cell line such as P3×63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO211, PRO228, PRO538, PRO172 or PRO182. Determination of “positive” hybridoma cells secreting the desired monoclonal antibodies against PRO211, PRO228, PRO538, PRO172 or PRO182 is within the skill in the art.

The positive hybridoma cells can be injected intraperitoneally into syngeneic Balb/c mice to produce ascites containing the anti-PRO211, anti-PRO228, anti-PRO538, anti-PRO172 or anti-PRO182 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 7

### Purification of PRO211, PRO228, PRO538, PRO172 or PRO182 Polypeptides Using Specific Antibodies

Native or recombinant PRO211, PRO228, PRO538, PRO172 or PRO182 polypeptides may be purified by a variety of standard techniques in the art of protein purification. For example, pro-PRO211, pro-PRO228, pro-PRO538, pro-PRO172 or pro-PRO182 polypeptide, mature PRO211, PRO228, PRO538, PRO172 or PRO182 polypeptide, or pre-PRO211, pre-PRO228, pre-PRO538, pre-PRO172 or pre-PRO182 polypeptide is purified by immunoaffinity chromatography using antibodies specific for the PRO211, PRO228, PRO538, PRO172 or PRO182 polypeptide of interest. In general, an immunoaffinity column is constructed by covalently coupling the anti-PRO211, anti-PRO228, anti-PRO538, anti-PRO172 or anti-PRO182 polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of the PRO211, PRO228, PRO538, PRO172 or PRO182 polypeptide by preparing a fraction from cells containing the PRO211, PRO228, PRO538, PRO172 or PRO182 polypeptide in a soluble form. This preparation is derived by solubilization of the whole cell or of a subcellular fraction obtained via differential centrifugation by the addition of detergent or by other methods well known in the art. Alternatively, soluble PRO211, PRO228, PRO538, PRO172 or PRO182 polypeptide containing a signal sequence may be secreted in useful quantity into the medium in which the cells are grown.

A soluble PRO211, PRO228, PRO538, PRO172 or PRO182 polypeptide-containing preparation is passed over the immunoaffinity column, and the column is washed under conditions that allow the preferential absorbance of the PRO211, PRO228, PRO538, PRO172 or PRO182 polypeptide (e.g., high ionic strength buffers in the presence of detergent). Then, the column is eluted under conditions that disrupt antibody/PRO211, antibody/PRO228, antibody/PRO538, antibody/PRO172 or antibody/PRO182 polypeptide binding (e.g., a low pH buffer such as approximately pH 2-3, or a high concentration of a chaotrope such as urea or thiocyanate ion), and the PRO211, PRO228, PRO538, PRO172 or PRO182 polypeptide is collected.

### Example 8

### Drug Screening

This invention is particularly useful for screening compounds by using PRO211, PRO228, PRO538, PRO172 or PRO182 polypeptides or a binding fragment thereof in any of a variety of drug screening techniques. The PRO211, PRO228, PRO538, PRO172 or PRO182 polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO211, PRO228, PRO538, PRO172 or PRO182 polypeptide or fragment. Drugs are screened against such transformed cells in competitive binding assays. Such cells, either in viable or fixed form, can be used for standard binding assays. One may measure, for example, the formation of complexes between a PRO211, PRO228, PRO538, PRO172 or PRO182 polypeptide or a fragment and the agent being tested. Alternatively, one can examine the diminution in complex formation between the PRO211, PRO228, PRO538, PRO172 or PRO182 polypeptide and its target cell or target receptors caused by the agent being tested.

Thus, the present invention provides methods of screening for drugs or any other agents which can affect a PRO211, PRO228, PRO538, PRO172 or PRO182 polypeptide-associated disease or disorder. These methods comprise contacting such an agent with a PRO211, PRO228, PRO538, PRO172 or PRO182 polypeptide or fragment thereof and assaying (i) for the presence of a complex between the agent and the PRO211, PRO228, PRO538, PRO172 or PRO182 polypeptide or fragment, or (ii) for the presence of a complex between the PRO211, PRO228, PRO538, PRO172 or PRO182 polypeptide or fragment and the cell, by methods well known in the art. In such competitive binding assays, the PRO211, PRO228, PRO538, PRO172 or PRO182 polypeptide or fragment is typically labeled. After suitable incubation, the free PRO211, PRO228, PRO538, PRO172 or PRO182 polypeptide or fragment is separated from that present in bound form, and the amount of free or uncomplexed label is a measure of the ability of the particular agent to bind to the PRO211, PRO228, PRO538, PRO172 or PRO182 polypeptide or to interfere with the PRO211, PRO228, PRO538, PRO172 or PRO182 polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO211, PRO228, PRO538, PRO172 or PRO182 polypeptide, the peptide test compounds are reacted with the PRO211, PRO228, PRO538, PRO172 or PRO182 polypeptide and washed. Bound PRO211, PRO228, PRO538, PRO172 or PRO182 polypeptide is detected by methods well known in the art. Purified PRO211, PRO228, PRO538, PRO172 or PRO182 polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding a PRO211, PRO228, PRO538, PRO172 or PRO182 polypeptide specifically compete with a test compound for binding to the PRO211, PRO228, PRO538, PRO172 or PRO182 polypeptide or fragments thereof. In this manner, the antibodies can be used to detect the presence of any peptide which shares one or more antigenic determinants with a PRO211, PRO228, PRO538, PRO172 or PRO182 polypeptide.

### Example 9

### Rational Drug Design

The goal of rational drug design is to produce structural analogs of a biologically active polypeptide of interest (i.e., a PRO211, PRO228, PRO538, PRO172 or PRO182 polypeptide) or of small molecules with which they interact, e.g., agonists, antagonists, or inhibitors. Any of these examples can be used to fashion drugs which are more active or stable forms of the PRO211, PRO228, PRO538, PRO172 or PRO182 polypeptide or which enhance or interfere with the function of the PRO211, PRO228, PRO538, PRO172 or PRO182 polypeptide in vivo (c.f., Hodgson, Bio/Technology, 9:19-21 (1991)).

In one approach, the three-dimensional structure of the PRO211, PRO228, PRO538, PRO172 or PRO182 polypeptide, or of a PRO211, PRO228, PRO538, PRO172 or PRO182 polypeptide-inhibitor complex, is determined by x-ray crystallography, by computer modeling or, most typically, by a combination of the two approaches. Both the shape and charges of the PRO211, PRO228, PRO538, PRO172 or PRO182 polypeptide must be ascertained to elucidate the structure and to determine active site(s) of the molecule. Less often, useful information regarding the structure of the PRO211, PRO228, PRO538, PRO172 or PRO182 polypeptide may be gained by modeling based on the structure of homologous proteins. In both cases, relevant structural information is used to design analogous PRO211, PRO228, PRO538, PRO172 or PRO182 polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO211, PRO228, PRO538, PRO172 or PRO182 polypeptide may be made available to perform such analytical studies as X-ray crystallography. In addition, knowledge of the PRO211, PRO228, PRO538, PRO172 or PRO182 polypeptide amino acid sequence provided herein will provide guidance to those employing computer modeling techniques in place of or in addition to x-ray crystallography.

### Example 10

### In Vitro Antitumor Assay

The antiproliferative activity of the PRO211, PRO228, PRO538, PRO172 and PRO182 polypeptides was determined in the investigational, disease-oriented in vitro anti-cancer drug discovery assay of the National Cancer Institute (NCI), using a sulforhodamine B (SRB) dye binding assay essentially as described by Skehan et al., J. Natl. Cancer Inst. 82:1107-1112(1990). The 60 tumor cell lines employed in this study (“the NCI panel”), as well as conditions for their maintenance and culture in vitro have been described by Monks et al., J. Natl. Cancer Inst., 83:757-766 (1991). The purpose of this screen is to initially evaluate the cytotoxic and/or cytostatic activity of the test compounds against different types of tumors (Monks et al., supra; Boyd, Cancer: Princ. Pract. Oncol. Update, 3(10):1-12 [1989]).

Cells from approximately 60 human tumor cell lines were harvested with trypsin/EDTA (Gibco), washed once, resuspended in IMEM and their viability was determined. The cell suspensions were added by pipet (100 μl volume) into separate 96-well microtiter plates. The cell density for the 6-day incubation was less than for the 2-day incubation to prevent overgrowth. Inoculates were allowed a preincubation period of 24 hours at 37° C. for stabilization. Dilutions at twice the intended test concentration were added at time zero in 100 μl aliquots to the microtiter plate wells (1:2 dilution). Test compounds were evaluated at five half-log dilutions (1000 to 100,000-fold). Incubations took place for two days and six days in a 5% CO₂ atmosphere and 100% humidity.

After incubation, the medium was removed and the cells were fixed in 0.1 ml of 10% trichloroacetic acid at 40° C. The plates were rinsed five times with deionized water, dried, stained for 30 minutes with 0.1 ml of 0.4% sulforhodamine B dye (Sigma) dissolved in 1% acetic acid, rinsed four times with 1% acetic acid to remove unbound dye, dried, and the stain was extracted for five minutes with 0.1 ml of 10 mM Tris base [tris(hydroxymethyl)aminomethane], pH 10.5. The absorbance (OD) of sulforhodamine B at 492 nm was measured using a computer-interfaced, 96-well microtiter plate reader.

A test sample is considered positive if it shows at least 40% growth inhibitory effect at one or more concentrations. The results are shown in the following Tables 5-9, where the tumor cell type abbreviations are as follows:

NSCL=non-small cell lung carcinoma; CNS=central nervous system

TABLE 5

| Compound | Concentration | Days | Tumor Cell Type | Designation |
|---|---|---|---|---|
| PRO211 | 0.65 nM | 6 | NSCL | HOP62 |
| PRO211 | 6.50 nM | 6 | Leukemia | RPMI-8226 |
| PRO211 | 6.50 nM | 6 | Leukemia | HL-60 (TB) |
| PRO211 | 6.50 nM | 6 | NSCL | NCI-H522 |
| PRO211 | 6.50 nM | 6 | CNS | SF-539 |
| PRO211 | 6.50 nM | 6 | Melanoma | LOX IMVI |
| PRO211 | 6.50 nM | 6 | Breast | MDA-MB-435 |
| PRO211 | 3.90 nM | 6 | Leukemia | MOLT-4 |
| PRO211 | 3.90 nM | 6 | CNS | U251 |
| PRO211 | 3.90 nM | 6 | Breast | MCF7 |
| PRO211 | 39.00 nM | 6 | Leukemia | HT-60 (TB) |
| PRO211 | 39.00 nM | 6 | Leukemia | MOLT-4 |
| PRO211 | 39.00 nM | 6 | NSCL | EKVX |
| PRO211 | 39.00 nM | 6 | NSCL | NCI-H23 |
| PRO211 | 39.00 nM | 6 | NSCL | NCI-H322M |
| PRO211 | 39.00 nM | 6 | NSCL | NCI-H460 |
| PRO211 | 39.00 nM | 6 | Colon | HCT-116 |
| PRO211 | 39.00 nM | 6 | Colon | HT29 |
| PRO211 | 39.00 nM | 6 | CNS | SF-268 |
| PRO211 | 39.00 nM | 6 | CNS | SF-295 |
| PRO211 | 39.00 nM | 6 | CNS | SNB-19 |
| PRO211 | 39.00 nM | 6 | CNS | U251 |
| PRO211 | 39.00 nM | 6 | Melanoma | LOX IMVI |
| PRO211 | 39.00 nM | 6 | Melanoma | SK-MEL-5 |
| PRO211 | 39.00 nM | 6 | Melanoma | UACC-257 |
| PRO211 | 39.00 nM | 6 | Melanoma | UACC-62 |
| PR0211 | 39.00 nM | 6 | Ovarian | OVCAR-8 |
| PRO211 | 39.00 nM | 6 | Renal | RXF 393 |
| PRO211 | 39.00 nM | 6 | Breast | MCF7 |
| PRO211 | 39.00 nM | 6 | Breast | NCI/ADR- |
| | | | | REHS 578T |
| PRO211 | 39.00 nM | 6 | Breast | T-47D |
| PRO211 | 39.00 nM | 2 | Leukemia | HL-60 (TB) |
| PRO211 | 39.00 nM | 2 | Leukemia | SR |
| PRO211 | 39.00 nM | 2 | NSCL | NCI-H23 |
| PRO211 | 39.00 nM | 2 | Colon | HCT-116 |
| PRO211 | 39.00 nM | 2 | Melanoma | LOX-IMVI |
| PRO211 | 39.00 nM | 2 | Melanoma | SK-MEL-5 |
| PRO211 | 39.00 nM | 2 | Breast | T-47D |

TABLE 6

| Compound | Concentration | Days | Tumor Cell Type | Designation |
|---|---|---|---|---|
| PRO228 | 0.77 nM | 6 | Leukemia | MOLT-4 |
| PRO228 | 0.77 nM | 6 | NSCL | EKVX |
| PRO228 | 0.77 nM | 6 | Colon | KM12 |
| PRO228 | 0.77 nM | 6 | Melanoma | UACC-62 |
| PRO228 | 0.77 nM | 6 | Ovarian | OVCAR-3 |
| PRO228 | 0.77 nM | 6 | Renal | TK10 |
| PRO228 | 0.77 nM | 6 | Renal | SN12C |
| PRO228 | 0.77 nM | 6 | Breast | MCF7 |
| PRO228 | 7.77 nM | 6 | Leukemia | CCRF-CEM |
| PRO228 | 7.77 nM | 6 | Leukemia | HL-60 (TB) |
| PRO228 | 7.77 nM | 6 | Colon | COLO 205 |
| PRO228 | 7.77 nM | 6 | Colon | HCT-15 |
| PRO228 | 7.77 nM | 6 | Colon | KM12 |
| PRO228 | 7.77 nM | 6 | CNS | SF-268 |
| PRO228 | 7.77 nM | 6 | CNS | SNB-75 |
| PRO228 | 7.77 nM | 6 | Melanoma | LOX-IMVI |
| PRO228 | 7.77 nM | 6 | Melanoma | SK-MEL2 |
| PRO228 | 7.77 nM | 6 | Melanoma | UACC-257 |
| PRO228 | 7.77 nM | 6 | Ovarian | IGROV1 |
| PRO228 | 7.77 nM | 6 | Ovarian | OVCAR-4 |
| PRO228 | 7.77 nM | 6 | Ovarian | OVCAR-5 |
| PRO228 | 7.77 nM | 6 | Ovarian | OVCAR-8 |
| PRO228 | 7.77 nM | 6 | Renal | 786-0 |
| PRO228 | 7.77 nM | 6 | Renal | CAKI-1 |
| PRO228 | 7.77 nM | 6 | Renal | RXF 393 |
| PRO228 | 7.77 nM | 6 | Renal | TK-10 |
| PRO228 | 7.77 nM | 6 | Renal | UO-31 |
| PRO228 | 7.77 nM | 6 | Prostate | PC-3 |
| PRO228 | 7.77 nM | 6 | Prostate | DU-145 |
| PRO228 | 7.77 nM | 6 | Breast | MCF7 |
| PRO228 | 7.77 nM | 6 | Breast | NCI/ADR- |
| | | | | REHS 578T |
| PRO228 | 7.77 nM | 6 | Breast | MDA-MB- |
| | | | | 435MDA-N |
| PRO228 | 7.77 nM | 6 | Breast | T-47D |

TABLE 7

| Compound | Concentration | Days | Tumor Cell Type | Designation |
|---|---|---|---|---|
| PRO538 | | 2 | Leukemia | SR |
| PRO538 | | 2 | CNS | SF-539 |
| PRO538 | | 2 | Renal | RXF 393 |
| PRO538 | | 6 | Leukemia | HL-60 (TB) |
| PRO538 | | 6 | NSCL | EKVX |
| PRO538 | | 6 | NSCL | HOP* |
| PRO538 | | 6 | NSCL | NCI-H23* |
| PRO538 | | 6 | NSCL | NCI-H322M |
| PRO538 | | 6 | NSCL | NCI-H460* |
| PRO538 | | 6 | Colon | HCC-2998 |
| PRO538 | | 6 | Colon | HCT-116 |
| PRO538 | | 6 | Colon | HT29 |
| PRO538 | | 6 | CNS | SF-268* |
| PRO538 | | 6 | CNS | SF-295 |
| PRO538 | | 6 | CNS | SNB-19 |
| PRO538 | | 6 | CNS | U251 |
| PRO538 | | 6 | Melanoma | LOX IMVI |
| PRO538 | | 6 | Melanoma | SK-MEL-2 |
| PRO538 | | 6 | Melanoma | SK-MEL-28 |
| PRO538 | | 6 | Melanoma | SK-MEL-5 |
| PRO538 | | 6 | Melanoma | UACC-25* |
| PRO538 | | 6 | Melanoma | UACC-62 |
| PRO538 | | 6 | Ovarian | OVCAR-5* |
| PRO538 | | 6 | Ovarian | OVCAR-8* |
| PRO538 | | 6 | Renal | 768-0 |
| PRO538 | | 6 | Renal | ACHN |
| PRO538 | | 6 | Renal | CAKI-1** |
| PRO538 | | 6 | Renal | RXF 393* |
| PRO538 | | 6 | Renal | SN12C |
| PRO538 | | 6 | Renal | TK-10 |
| PRO538 | | 6 | Prostate | PC-3 |
| PRO538 | | 6 | Prostate | DU-145* |
| PRO538 | | 6 | Breast | MDA-MB-231 |
| PRO538 | | 6 | Breast | HS 578T* |
| PRO538 | | 6 | Breast | ST-549* |
| PRO538 | | 6 | Breast | T-47D |

*cytotoxic effect
**cytostatic effect

TABLE 8

| Compound | Concentration | Days | Tumor Cell Type | Designation |
|---|---|---|---|---|
| PRO172 | 1.25 nM | 2 | Breast | T-470 |
| PRO172 | 1.25 nM | 6 | NSCL | NCI-H460 |
| PRO172 | 1.25 nM | 6 | Colon | KM12 |
| PRO172 | 1.25 nM | 6 | CNS | SF-295 |
| PRO172 | 1.25 nM | 6 | Melanoma | UACC-62 |
| PRO172 | 1.25 nM | 2 | Breast | MDA-MB- |
| | | | | 231/ATCC |
| PRO172 | 1.25 nM | 6 | Leukemia | CCRF-CEM |
| PRO172 | 1.25 nM | 6 | Leukemia | MOLT4 |
| PRO172 | 1.25 nM | 6 | NSCL | NCI-H460 |
| PRO172 | 1.25 nM | 6 | Colon | HCT-116 |
| PRO172 | 1.25 nM | 6 | Colon | HT29 |
| PRO172 | 1.25 nM | 6 | CNS | SF-295 |
| PRO172 | 1.25 nM | 6 | CNS | U251 |
| PRO172 | 1.25 nM | 6 | Melanoma | LOX IMVI |
| PRO172 | 1.25 nM | 6 | Melanoma | UACC-62 |
| PRO172 | 1.25 nM | 6 | Ovarian | OVCAR-8 |
| PRO172 | 1.25 nM | 6 | Renal | RXF 393 |
| PRO172 | 1.25 nM | 6 | Breast | T-470 |

TABLE 9

| Compound | Concentration | Days | Tumor Cell Type | Designation |
|---|---|---|---|---|
| PRO182 | 0.85 nM | 2 | Leukemia | K-562 |
| PRO182 | 0.85 nM | 6 | Leukemia | HL-60 (TB) |
| PRO182 | 6.70 nM | 6 | Ovarian | OVCAR-5 |
| PRO182 | 6.70 nM | 6 | Leukemia | HL-60 (TB) |
| PRO182 | 6.70 nM | 6 | Colon | COLO205 |
| PRO182 | 6.70 nM | 6 | Melanoma | LOX IMVI |
| PRO182 | 67.0 nM | 2 | NSCL | EKVX |
| PRO182 | 67.0 nM | 2 | NSCL | NCI-H226 |
| PRO182 | 67.0 nM | 2 | Ovarian | IGROV1 |
| PRO182 | 67.0 nM | 2 | Ovarian | N3VCAR3 |
| PRO182 | 67.0 nM | 2 | Breast | HS378T |
| PRO182 | 67.0 nM | 2 | Breast | T47D |
| PRO182 | 67.0 nM | 6 | Leukemia | CCRF-CEM |
| PRO182 | 67.0 nM | 6 | Leukemia | HL-60 (TB) |
| PRO182 | 67.0 nM | 6 | Leukemia | MOLT4 |
| PRO182 | 67.0 nM | 6 | Leukemia | SR |
| PRO182 | 67.0 nM | 6 | NSCL | NCI-H23 |
| PRO182 | 67.0 nM | 6 | NSCL | NCI-H460 |
| PRO182 | 67.0 nM | 6 | CNS | U251 |
| PRO182 | 67.0 nM | 6 | Melanoma | UACC-257 |
| PRO182 | 67.0 nM | 6 | Melanoma | UACC-62 |
| PRO182 | 67.0 nM | 6 | Renal | RXF-393 |
| PRO182 | 42.0 nM | 6 | Leukemia | MOLT4 |
| PRO182 | 42.0 nM | 6 | Leukemia | SR |
| PRO182 | 42.0 nM | 6 | NSCL | A549/ATCC |
| PRO182 | 42.0 nM | 6 | NSCL | NCI/H322M |
| PRO182 | 42.0 nM | 6 | Colon | HCT-18 |
| PRO182 | 42.0 nM | 6 | Melanoma | UACC-257 |
| PRO182 | 42.0 nM | 6 | Melanoma | USCC-62 |
| PRO182 | 42.0 nM | 2 | Renal | RXF 393 |

Deposit of Material

The following materials have been deposited with the American Type Culture Collection, 10801 University Blvd., Manassas, Va. 20110-2209, USA (ATCC):

| Material | ATCC Dep. No. | Deposit Date |
|---|---|---|
| DNA32292-1131 | 209258 | September 16, 1997 |
| DNA33092-1202 | 209420 | October 28, 1997 |
| DNA48613-1268 | 209752 | April 7, 1998 |
| DNA35916-1161 | 209419 | October 28, 1997 |
| DNA27865-1091 | 209296 | September 23, 1997 |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposits will be made available by ATCC under the terms of the Budapest Treaty, and subject to an agreement between Genentech, Inc., and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. § 122 and the Commissioner's rules pursuant thereto (including 37 CFR § 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. A composition of matter useful for the inhibition of neoplastic cell growth, said composition comprising an effective amount of a PRO211, PRO228, PRO538, PRO172 or PRO182 polypeptide, or an agonist thereof, in admixture with a pharmaceutically acceptable carrier.

2. The composition of matter of claim 1 comprising a growth inhibitory amount of a PRO211, PRO228, PRO538, PRO172 or PRO182 polypeptide, or an agonist thereof.

3. The composition of matter of claim 1 comprising a cytotoxic amount of a PRO211, PRO228, PRO538, PRO172 or PRO182 polypeptide, or an agonist thereof.

4. The composition of matter of claim 1 additionally comprising a further growth inhibitory agent, cytotoxic agent or chemotherapeutic agent.

5. The composition of matter of claim 1, wherein said PRO211 polypeptide comprises at least about 80% amino acid sequence identity to (a) residues 1 or about 25 to 353 of the PRO211 polypeptide shown in FIG. 2 (SEQ ID NO:2) or (b) X to 353 of the PRO211 polypeptide shown in FIG. 2 (SEQ ID NO:2), wherein X is any amino acid residue from 20 to 29 of FIG. 2 (SEQ ID NO:2).

6. The composition of matter of claim 5, wherein said PRO211 polypeptide comprises the amino acid sequence shown in FIG. 2 (SEQ ID NO:2).

7. The composition of matter of claim 1, wherein said PRO228 polypeptide comprises at least about 80% amino acid sequence identity to (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7) or (c) 1 or about 20 to X of FIG. 4 (SEQ ID NO:7), wherein X is any amino acid from amino acid 425 to amino acid 434 of FIG. 4 (SEQ ID NO:7).

8. The composition of matter of claim 7, wherein said PRO228 polypeptide comprises the amino acid sequence shown in FIG. 4 (SEQ ID NO:7).

9. The composition of matter of claim 1, wherein said PRO538 polypeptide comprises at least about 80% amino acid sequence identity to (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16) or (c) 1 or about 27 to X of FIG. 6 (SEQ ID NO:16), wherein X is any amino acid from amino acid 374 to amino acid 383 of FIG. 6 (SEQ ID NO:16).

10. The composition of matter of claim 9, wherein said PRO538 polypeptide comprises the amino acid sequence shown in FIG. 6 (SEQ ID NO:16).

11. The composition of matter of claim 1, wherein said PRO172 polypeptide comprises at least about 80% amino acid sequence identity to (a) residues 1 or about 22 to 723 of the PRO172 polypeptide shown in FIG. 8 (SEQ ID NO:21), (b) X to 723 of the PRO172 polypeptide shown in FIG. 8 (SEQ ID NO:21), wherein X is any amino acid residue from 17 to 26 of FIG. 8 (SEQ ID NO:21) or (c) 1 or about 22 to X of FIG. 8 (SEQ ID NO:21), wherein X is any amino acid from amino acid 543 to amino acid 552 of FIG. 8 (SEQ ID NO:21).

12. The composition of matter of claim 11, wherein said PRO172 polypeptide comprises the amino acid sequence shown in FIG. 8 (SEQ ID NO:21).

13. The composition of matter of claim 1, wherein said PRO182 polypeptide comprises at least about 80% amino acid sequence identity to (a) residues 1 or about 19 to 135 of the PRO182 polypeptide shown in FIG. 10 (SEQ ID NO:26) or (b) X to 135 of the PRO182 polypeptide shown in FIG. 10 (SEQ ID NO:26), wherein X is any amino acid residue from 14 to 23 of FIG. 10 (SEQ ID NO:26).

14. The composition of matter of claim 13, wherein said PRO182 polypeptide comprises the amino acid sequence shown in FIG. 10 (SEQ ID NO:26).

15. A composition of matter useful for the treatment of a tumor in a mammal, said composition comprising a therapeutically effective amount of a PRO211, PRO228, PRO538, PRO172 or PRO182 polypeptide, or an agonist thereof.

16. The composition of matter of claim 15, wherein said tumor is a cancer.

17. The composition of matter of claim 16, wherein the cancer is selected from the group consisting of breast cancer, ovarian cancer, renal cancer, colorectal cancer, uterine cancer, prostate cancer, lung cancer, bladder cancer, central nervous system cancer, melanoma and leukemia.

18. The composition of matter of claim 15, wherein said PRO211 polypeptide comprises at least about 80% amino acid sequence identity to (a) residues 1 or about 25 to 353 of the PRO211 polypeptide shown in FIG. 2 (SEQ ID NO:2) or (b) X to 353 of the PRO211 polypeptide shown in FIG. 2 (SEQ ID NO:2), wherein X is any amino acid residue from 20 to 29 of FIG. 2 (SEQ ID NO:2).

19. The composition of matter of claim 18, wherein said PRO211 polypeptide comprises the amino acid sequence shown in FIG. 2 (SEQ ID NO:2).

20. The composition of matter of claim 15, wherein said PRO228 polypeptide comprises at least about 80% amino acid sequence identity to (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7) or (c) 1 or about 20 to X of FIG. 4 (SEQ ID NO:7), wherein X is any amino acid from amino acid 425 to amino acid 434 of FIG. 4 (SEQ ID NO:7).

21. The composition of matter of claim 20, wherein said PRO228 polypeptide comprises the amino acid sequence shown in FIG. 4 (SEQ ID NO:7).

22. The composition of matter of claim 15, wherein said PRO538 polypeptide comprises at least about 80% amino acid sequence identity to (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16) or (c) 1 or about 27 to X of FIG. 6 (SEQ ID NO:16), wherein X is any amino acid from amino acid 374 to amino acid 383 of FIG. 6 (SEQ ID NO:16).

23. The composition of matter of claim 22, wherein said PRO538 polypeptide comprises the amino acid sequence shown in FIG. 6 (SEQ ID NO:16).

24. The composition of matter of claim 15, wherein said PRO172 polypeptide comprises at least about 80% amino acid sequence identity to (a) residues 1 or about 22 to 723 of the PRO172 polypeptide shown in FIG. 8 (SEQ ID NO:21), (b) X to 723 of the PRO172 polypeptide shown in FIG. 8 (SEQ ID NO:21), wherein X is any amino acid residue from 17 to 26 of FIG. 8 (SEQ ID NO:21) or (c) 1 or about 22 to X of FIG. 8 (SEQ ID NO:21), wherein X is any amino acid from amino acid 543 to amino acid 552 of FIG. 8 (SEQ ID NO:21).

25. The composition of matter of claim 24, wherein said PRO172 polypeptide comprises the amino acid sequence shown in FIG. 8 (SEQ ID NO:21).

26. The composition of matter of claim 15, wherein said PRO182 polypeptide comprises at least about 80% amino acid sequence identity to (a) residues 1 or about 19 to 135 of the PRO182 polypeptide shown in FIG. 10 (SEQ ID NO:26) or (b) X to 135 of the PRO182 polypeptide shown in FIG. 10 (SEQ ID NO:26), wherein X is any amino acid residue from 14 to 23 of FIG. 10 (SEQ ID NO:26).

27. The composition of matter of claim 26, wherein said PRO182 polypeptide comprises the amino acid sequence shown in FIG. 10 (SEQ ID NO:26).

28. A method for inhibiting the growth of a tumor cell comprising exposing said tumor cell to an effective amount of a PRO211, PRO228, PRO538, PRO172 or PRO182 polypeptide, or an agonist thereof.

29. The method of claim 28, wherein said PRO211 polypeptide comprises at least about 80% amino acid sequence identity to (a) residues 1 or about 25 to 353 of the PRO211 polypeptide shown in FIG. 2 (SEQ ID NO:2) or (b) X to 353 of the PRO211 polypeptide shown in FIG. 2 (SEQ ID NO:2), wherein X is any amino acid residue from 20 to 29 of FIG. 2 (SEQ ID NO:2).

30. The method of claim 29, wherein said PRO211 polypeptide comprises the amino acid sequence shown in FIG. 2 (SEQ ID NO:2).

31. The method of claim 28, wherein said PRO228 polypeptide comprises at least about 80% amino acid sequence identity to (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7) or (c) 1 or about 20 to X of FIG. 4 (SEQ ID NO:7), wherein X is any amino acid from amino acid 425 to amino acid 434 of FIG. 4 (SEQ ID NO:7).

32. The method of claim 31, wherein said PRO228 polypeptide comprises the amino acid sequence shown in FIG. 4 (SEQ ID NO:7).

33. The method of claim 28, wherein said PRO538 polypeptide comprises at least about 80% amino acid sequence identity to (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16) or (c) 1 or about 27 to X of FIG. 6 (SEQ ID NO:16), wherein X is any amino acid from amino acid 374 to amino acid 383 of FIG. 6 (SEQ ID NO:16).

34. The method of claim 33, wherein said PRO538 polypeptide comprises the amino acid sequence shown in FIG. 6 (SEQ ID NO:16).

35. The method of claim 28, wherein said PRO172 polypeptide comprises at least about 80% amino acid sequence identity to (a) residues 1 or about 22 to 723 of the PRO172 polypeptide shown in FIG. 8 (SEQ ID NO:21), (b) X to 723 of the PRO173 polypeptide shown in FIG. 8 (SEQ ID NO:21), wherein X is any amino acid residue from 17 to 26 of FIG. 8 (SEQ ID NO:21) or (c) 1 or about 22 to X of FIG. 8 (SEQ ID NO:21), wherein X is any amino acid from amino acid 543 to amino acid 552 of FIG. 8 (SEQ ID NO:21).

36. The method of claim 35, wherein said PRO172 polypeptide comprises the amino acid sequence shown in FIG. 8 (SEQ ID NO:21).

37. The method of claim 28, wherein said PRO182 polypeptide comprises at least about 80% amino acid sequence identity to (a) residues 1 or about 19 to 135 of the PRO182 polypeptide shown in FIG. 10 (SEQ ID NO:26) or (b) X to 135 of the PRO182 polypeptide shown in FIG. 10 (SEQ ID NO:26), wherein X is any amino acid residue from 14 to 23 of FIG. 10 (SEQ ID NO:26).

38. The method of claim 37, wherein said PRO182 polypeptide comprises the amino acid sequence shown in FIG. 10 (SEQ ID NO:26).

39. The method of claim 28, wherein said agonist is an anti-PRO211, anti-PRO228, anti-PRO538, anti-PRO172 or anti-PRO182 agonist antibody.

40. The method of claim 28, wherein said agonist is a small molecule mimicking the biological activity of a PRO211, PRO228, PRO538, PRO172 or PRO182 polypeptide.

41. The method of claim 28, wherein said step of exposing occurs in vitro.

42. The method of claim 28, wherein said step of exposing occurs in vivo.

43. An article of manufacture comprising:
a container; and
a composition comprising an active agent contained within the container; wherein said active agent in the composition is a PRO211, PRO228, PRO538, PRO172 or PRO182 polypeptide, or an agonist thereof.

44. The article of manufacture of claim 43, wherein said PRO211 polypeptide comprises at least about 80% amino acid sequence identity to (a) residues 1 or about 25 to 353 of the PRO211 polypeptide shown in FIG. 2 (SEQ ID NO:2) or (b) X to 353 of the PRO211 polypeptide shown in FIG. 2 (SEQ ID NO:2), wherein X is any amino acid residue from 20 to 29 of FIG. 2 (SEQ ID NO:2).

45. The article of manufacture of claim 44, wherein said PRO211 polypeptide comprises the amino acid sequence shown in FIG. 2 (SEQ ID NO:2).

46. The article of manufacture of claim 43, wherein said PRO228 polypeptide comprises at least about 80% amino acid sequence identity to (a) residues 1 or about 20 to 690 of the PRO228 polypeptide shown in FIG. 4 (SEQ ID NO:7), (b) X to 690 of the PRO228 polypeptide shown in FIG. 4 (SEQ ID NO:7), wherein X is any amino acid residue from 15 to 24 of FIG. 4 (SEQ ID NO:7) or (c) 1 or about 20 to X of FIG. 4 (SEQ ID NO:7), wherein X is any amino acid from amino acid 425 to amino acid 434 of FIG. 4 (SEQ ID NO:7).

47. The article of manufacture of claim 46, wherein said PRO228 polypeptide comprises the amino acid sequence shown in FIG. 4 (SEQ ID NO:7).

48. The article of manufacture of claim 43, wherein said PRO538 polypeptide comprises at least about 80% amino acid sequence identity to (a) residues 1 or about 27 to 400 of the PRO538 polypeptide shown in FIG. 6 (SEQ ID NO:16), (b) X to 400 of the PRO538 polypeptide shown in FIG. 6 (SEQ ID NO:16), wherein X is any amino acid residue from 22 to 31 of FIG. 6 (SEQ ID NO:16) or (c) 1 or about 27 to X of FIG. 6 (SEQ ID NO:16), wherein X is any amino acid from amino acid 374 to amino acid 383 of FIG. 6 (SEQ ID NO:16).

49. The article of manufacture of claim 48, wherein said PRO538 polypeptide comprises the amino acid sequence shown in FIG. 6 (SEQ ID NO:16).

50. The article of manufacture of claim 43, wherein said PRO172 polypeptide comprises at least about 80% amino acid sequence identity to (a) residues 1 or about 22 to 723 of the PRO172 polypeptide shown in FIG. 8 (SEQ ID NO:21), (b) X to 723 of the PRO172 polypeptide shown in FIG. 8 (SEQ ID NO:21), wherein X is any amino acid residue from 17 to 26 of FIG. 8 (SEQ ID NO:21) or (c) 1 or about 22 to X of FIG. 8 (SEQ ID NO:21), wherein X is any amino acid from amino acid 543 to amino acid 552 of FIG. 8 (SEQ ID NO:21).

51. The article of manufacture of claim 50, wherein said PRO172 polypeptide comprises the amino acid sequence shown in FIG. 8 (SEQ ID NO:21).

52. The article of manufacture of claim 43, wherein said PRO182 polypeptide comprises at least about 80% amino acid sequence identity to (a) residues 1 or about 19 to 135 of the PRO182 polypeptide shown in FIG. 10 (SEQ ID NO:26) or (b) X to 135 of the PRO182 polypeptide shown in FIG. 10 (SEQ ID NO:26), wherein X is any amino acid residue from 14 to 23 of FIG. 10 (SEQ ID NO:26).

53. The article of manufacture of claim 52, wherein said PRO182 polypeptide comprises the amino acid sequence shown in FIG. 10 (SEQ ID NO:26).

54. The article of manufacture of claim 43, wherein said agonist is an anti-PRO211, anti-PRO228, anti-PRO538, anti-PRO172 or anti-PRO182 agonist antibody.

55. The article of manufacture of claim 43, wherein said agonist is a small molecule mimicking the biological activity of a PRO211, PRO228, PRO538, PRO172 or PRO182 polypeptide.

56. The article of manufacture of claim 43, wherein said active agent is present in an amount that is effective for the treatment of tumor in a mammal.

57. The article of manufacture of claim 43, wherein said composition additionally comprises a further growth inhibitory agent, cytotoxic agent or chemotherapeutic agent.
